(19) United States
(12) Patent Application Publication
(10) Pub. No.: US 2024/0271132 A1
(43) Pub. Date: Aug. 15, 2024

(54) MIRNA INHIBITORS FOR PREVENTING AND TREATING ANEURYSMS, HYPERTENSION, ARDS AND OTHER DISEASES ASSOCIATED WITH ENDOTHELIAL DYSFUNCTION

(71) Applicant: The Regents of the University of California, Oakland, CA (US)

(72) Inventor: Hua Cai, Los Angeles, CA (US)

(21) Appl. No.: 18/289,418

(22) PCT Filed: May 9, 2022
(86) PCT No.: PCT/US22/28296

§ 371 Date: Nov. 3, 2023

Related U.S. Application Data
(60) Provisional application No. 63/185,788, filed on May 7, 2021.

(51) Int. Cl. C12N 15/113; A61K 38/44; A61K 45/06; A61P 9/10

(52) U.S. Cl. CPC C12N 15/113; A61K 38/44; A61K 45/06; A61P 9/10; C12Y 114/13039; C12N 2310/141; C12N 2310/315; C12N 2310/321; C12N 2310/322; C12N 2310/3231; C12N 2310/3233; C12N 2310/3515

(57) ABSTRACT

The present disclosure relates to pharmaceutical compositions comprising a miRNA inhibitor, as well as the method for using such pharmaceutical compositions.

### RELATED APPLICATIONS

This application claims the benefit of priority to U.S. Provisional Patent Application Ser. No. 63/185,788, filed May 7, 2021, the contents of which are hereby incorporated herein by reference in their entirety.

### GOVERNMENT SUPPORT

This invention was made with government support under Grant Number HL077440, awarded by the National Institutes of Health. The government has certain rights in the invention.

### BACKGROUND

Abdominal aortic aneurysm (AAA) is defined as an abdominal aortic dilation of over 3 cm in diameter, most commonly affecting the infrarenal segment. It is associated with high risk of mortality in the event of aneurysm rupture, leading to 150,000-200,000 deaths each year worldwide. The incidence of AAA is up to 9% in the overall population older than 65. While the most well-recognized risk factors for AAA include male gender and smoking, other risk factors have been implicated in AAA formation such as older age, family history, hypertension and hyperlipidemia. The mechanisms of AAA formation are complex, primarily involving inflammation and oxidative stress mediated matrix degradation and vascular remodeling, which result in expansion of abdominal aortas. Current clinical intervention to prevent rupture of large AAAs of over 5.5 cm in size is limited to surgical repair of expanding arteries or stent installation, with considerable risk of up to 5% mortality rate. There have been no oral medicines available to treat the smaller and growing aneurysms to prevent unpredictable sudden rupture and death.

Hypertension is a prevalent and severe cardiovascular disorder, affecting more than 30% of adult population worldwide. Despite existing treatments, many hypertensive patients are drug resistant in responding to the available treatment options of four classes of drugs. Hypertension is a major contributor to atherosclerotic coronary artery disease, associating with exceedingly high mortality. New therapeutic options are in urgent need for drug resistant hypertension.

Acute respiratory distress syndrome (ARDS) is another devastating, lethal clinical condition that is associated with endothelial dysfunction, a common mediator shared by development of aneurysm, hypertension, ARDS or other diseases associated with endothelial dysfunction. New therapeutic options are in urgent need for ARDS.

### SUMMARY OF THE INVENTION

In certain aspects, provided herein are pharmaceutical compositions comprising a miRNA inhibitor. Such inhibitors and their compositions are useful in medical treatment, such as the prevention, inhibition, reduction, and treatment of aneurysms, hypertension, ARDS, and other diseases associated with endothelial dysfunction.

The compositions and methods described herein can be used to prevent, inhibit, treat, or reduce aneurysms, hypertension, ARDS, and other diseases associated with endothelial dysfunction in a subject, comprising administering to the subject a pharmaceutical composition comprising a miRNA inhibitor comprising a nucleic acid sequence that binds to at least a portion of a miR-192-5p sequence or those miRNAs listed in Tables 1-4.

In certain embodiments, the pharmaceutical composition comprises a vector encoding the miRNA inhibitor.

In some embodiments, the miRNA inhibitor suppresses the function of the mature miR-192-5p or those miRNAs listed in Tables 1-4.

This invention also provides methods of preventing, inhibiting, treating, or reducing aneurysms, hypertension, ARDS, and other diseases associated with endothelial dysfunction in a subject, comprising administering (e.g., administered subcutaneously, parenterally) to the subject a pharmaceutical composition comprising an miRNA inhibitor comprising a nucleic acid sequence set forth in Tables 1-4.

In certain embodiments, the aneurysm is abdominal aortic aneurysm, cerebral aneurysm, or thoracic aortic aneurysm.

In certain embodiments, the hypertension is primary or secondary hypertension.

In certain embodiments, the ARDS is caused by trauma, bacterial or viral infections (e.g. SARS or COVID-19).

In certain embodiments, the other diseases associated with endothelial dysfunction refers to coronary artery disease, diabetic vascular complications, cerebral vascular disease, peripheral vascular disease, thromboembolic disease, ischemia reperfusion injury of the heart/myocardial infarction, or those listed later in below describing all relevant pathological conditions.

The methods described herein may further comprise conjointly administering to the subject an additional therapeutic agent (e.g., a folate compound, a calcium channel blocker, a reactive oxygen species/ROS scavenger).

Methods are also provided for reversing vascular remodeling, comprising administering to the subject a pharmaceutical composition comprising an miRNA inhibitor comprising a nucleic acid sequence that binds to at least a portion of a miR-192-5p sequence or those miRNAs listed in Tables 1-4, wherein vascular remodeling is characterized by inflammation, matrix degradation, adventitial hypertrophy, medial elastin degradation and flattening, and/or formation of intra-lumen thrombi.

In certain aspects, provided herein are methods of decreasing reactive oxygen species production, comprising administering to the subject a pharmaceutical composition comprising an miRNA inhibitor comprising a nucleic acid sequence that binds to at least a portion of a miR-192-5p sequence or those miRNAs listed in Tables 1-4, and wherein the miRNA inhibitor suppresses the function of the mature miR-192-5p or those miRNAs listed in Tables 1-4.

In certain aspects, provided herein are methods of decreasing reactive oxygen species production, comprising administering to the subject a pharmaceutical composition comprising an miRNA inhibitor comprising a nucleic acid sequence set forth in Tables 1-4.

In certain aspects, provided herein are methods of restoring endothelial nitric oxide synthase (eNOS) coupling activity, comprising administering to the subject a pharmaceutical composition comprising an miRNA inhibitor comprising a nucleic acid sequence that binds to at least a portion of a miR-192-5p sequence or those miRNAs listed in Tables 1-4, and wherein the miRNA inhibitor suppresses the function of the mature miR-192-5p or those miRNAs listed in Tables 1-4.

In certain aspects, provided herein are methods of restoring endothelial nitric oxide synthase (eNOS) coupling activity, comprising administering to the subject a pharmaceutical composition comprising an miRNA inhibitor comprising a nucleic acid sequence set forth in Tables 1-4.

In certain aspects, provided herein are methods of preserving nitric oxide (NO) bioavailability, comprising administering to the subject a pharmaceutical composition comprising an miRNA inhibitor comprising a nucleic acid sequence that binds to at least a portion of a miR-192-5p sequence or those miRNAs listed in Tables 1-4, and wherein the miRNA inhibitor suppresses the function of the mature miR-192-5p or those miRNAs listed in Tables 1-4.

In certain aspects, provided herein are methods of preserving nitric oxide (NO) bioavailability, comprising administering to the subject a pharmaceutical composition comprising an miRNA inhibitor comprising a nucleic acid sequence set forth in Tables 1-4.

Methods are also provided for treating or preventing aneurysm (abdominal aortic aneurysm (AAA), thoracic aortic aneurysm (TAA) or cerebral aneurysm), hypertension, acute respiratory distress syndrome (ARDS) and other diseases associated with endothelial dysfunction in a subject, comprising administering to the subject an miRNA inhibitor comprising a nucleic acid that is at least 50-100% identical to any one of SEQ ID NOs: 1-19.

Further, the methods described herein can be used to treat or prevent aneurysm (abdominal aortic aneurysm (AAA), thoracic aortic aneurysm (TAA) or cerebral aneurysm), hypertension, acute respiratory distress syndrome (ARDS) and other diseases associated with endothelial dysfunction in a subject, comprising administering to the subject a miRNA inhibitor comprising a nucleic acid sequence that binds to at least a portion of a miR-192-5p sequence or those miRNAs listed in Tables 1-4.

In certain embodiments, the miRNA inhibitor has at least 50%, at least 55%, at least 60%, at least 65%, at least 70%, at least 75%, at least 80%, at least 85%, 90%, at least 95%, at least 99%, or at least 100% complementarity to a portion of a miR-192-5p sequence or those miRNAs listed in Tables 1-4.

In some embodiments, the miRNA inhibitor suppresses the function of the mature miR-192-5p or those miRNAs listed in Tables 1-4.

In certain embodiments, the miRNA inhibitor comprises a nucleic acid sequence that is at least 50%, at least 55%, at least 60%, at least 65%, at least 70%, at least 75%, 80%, at least 90%, at least 95%, or at least 98% identical to any one of SEQ ID NOs: 1-19. In some embodiments, the miRNA inhibitor comprises a nucleic acid sequence that is any one of SEQ ID NOs: 1-19.

In some embodiments, the nucleic acid comprises any of the possible chemical modifications (e.g., a 2′-O-methylated nucleoside (2′OMe), a 2′-fluoro oligonucleotide (2′F), a 2′-O-methoxyethyl oligonucleotide (2′MOE), a phosphorodiamidate morpholino oligonucleotide (PMO), a peptide nucleic acid (PNA), a phosphorothioate bond (PS), a locked nucleic acid (LNA), a non-nucleotide N,N-diethyl-4-(4-nitronaphthalen-1-ylazo)-phenylamine (ZEN), a hydrophobic moiety, a naphthyl modifier, or a cholesterol moiety).

The nucleic acid may be modified by a HEN1 methytransferase.

In certain embodiments, the nucleic acid is complementary to any one of SEQ ID NOs: 20-38.

In certain embodiments, the miRNA inhibitor binds to a miRNA comprising a nucleic acid that is at least 50%, at least 55%, at least 60%, at least 65%, at least 70%, at least 75%, 80%, at least 90%, at least 95%, or at least 98% identical to any one SEQ ID NOs: 20-38. In some embodiments, the miRNA inhibitor binds to a miRNA comprising a nucleic acid that is any one SEQ ID NOs: 20-38.

In certain embodiments, the miRNA inhibitor is at least 8, 9, 10, 11, 12, 13, 14, 15, 16, 17, 18, 19, or 20 nucleotides in length.

In some embodiments, the miRNA inhibitor is no more than 32, 31, 30, 29, 28, 27, 26, 25, 24, 23, 22, 21, or 20 nucleotides in length.

### BRIEF DESCRIPTION OF THE DRAWINGS

FIG. 1A shows the upregulation of miR-192-5p in human AAA. The RNA was isolated from aortic samples of human AAA patients and control subjects (n=15 for each group). miR-192-5p was upregulated in aortic samples of human AAA patients comparing to the controls. Data are presented as Mean±SEM (n=15), **p<0.01 vs. control.

FIG. 1B shows that Hsa-miR-192-5p expression was upregulated in H₂O₂ stimulated HAECs. Human aortic endothelial cells (HAECs) were treated with hydrogen peroxide (H₂O₂, 100 μM, 24 h) and the RNA was isolated to detect miR-192-5p expression levels. Hsa-miR-192-5p expression was upregulated in H₂O₂ stimulated HAECs. Data are presented as Mean±SEM (n=4-6 per group), *p<0.05 vs. control

FIG. 2A shows that Hsa-miR-192-5p specific inhibitors attenuated miR-192-5p expression in H₂O₂ stimulated HAECs. Human aortic endothelial cells (HAECs) were treated with hydrogen peroxide (H₂O₂, 100 μM, 24 h) in the presence or absence of negative control miR inhibitors or Hsa-miR0192-5p inhibitors, and the RNA was isolated to detect miR-192-5p expression levels. Hsa-miR-192-5p specific inhibitors attenuated miR-192-5p expression in H₂O₂ stimulated HAECs. Data are presented as Mean±SEM (n=6), *p<0.05.

FIG. 2B shows that Hsa-miR-192-5p specific inhibitors markedly restored DHFR mRNA expression in H₂O₂ stimulated HAECs. Human aortic endothelial cells (HAECs) were treated with hydrogen peroxide (H₂O₂, 100 μM, 24 h) in the presence or absence of negative control miR inhibitors or Hsa-miR-192-5p inhibitors, and the RNA was isolated to detect DHFR mRNA expression levels. Hsa-miR-192-5p specific inhibitors markedly restored DHFR mRNA expression in H₂O₂ stimulated HAECs. Data are presented as Mean±SEM (n=4-6), **p<0.01, ***p<0.001.

FIG. 2C shows representative Western blots of endothelial DHFR protein expression with 3-actin serving as an internal control. Human aortic endothelial cells (HAECs) were treated with hydrogen peroxide (H₂O₂, 100 μM, 24 h) in the presence or absence of negative control miR inhibitors or Hsa-miR-192-5p inhibitors, and the protein was isolated to detect DHFR protein expression levels.

FIG. 2D shows that Hsa-miR-192-5p specific inhibitors markedly restored DHFR protein expression in H₂O₂ stimulated HAECs. Human aortic endothelial cells (HAECs) were treated with hydrogen peroxide (H₂O₂, 100 μM, 24 h) in the presence or absence of negative control miR inhibitors or Hsa-miR-192-5p inhibitors, and the protein was isolated to detect DHFR protein expression levels. Hsa-miR-192-5p specific inhibitors markedly restored DHFR protein expression in H₂O₂ stimulated HAECs. Data are presented as Mean±SEM (n=4), *p<0.05.

FIG. 3A shows the percentage of AAA in each experimental group of Ang II infused hph-1, hph-1-NOX1, hph-1-NOX2, hph-1-p47phox, and hph-1-NOX4 double mutant mice. Ang II was infused into hph-1, hph-1-NOX1, hph-1-NOX2, hph-1-p47phox, and hph-1-NOX4 double mutant animals prior to phenotyping for AAA. n=26-53 per group, ***p<0.001.

FIG. 3B shows mmu-miR-192-5p expression level in hph-1 and hph-1-NOX1 mice with and without Ang II infusion. Ang II was infused into hph-1, hph-1-NOX1, hph-1-NOX2, hph-1-p47phox, and hph-1-NOX4 double mutant animals prior to phenotyping for AAA and isolation of aortic endothelial cells for detection of miR-192-5p expression levels. Data are presented as Mean±SEM (n=5), *p<0.05, **p<0.01.

FIG. 3C shows the expression level of mmu-miR-192-5p in hph-1 and hph-1-NOX2 mice with and without Ang II infusion. Ang II was infused into hph-1, hph-1-NOX1, hph-1-NOX2, hph-1-p47phox, and hph-1-NOX4 double mutant animals prior to phenotyping for AAA and isolation of aortic endothelial cells for detection of miR-192-5p expression levels. Data are presented as Mean±SEM (n=6-7), *p<0.05.

FIG. 3D shows the expression level of mmu-miR-192-5p in hph-1 and hph-1-p47phox mice with and without Ang II infusion. Ang II was infused into hph-1, hph-1-NOX1, hph-1-NOX2, hph-1-p47phox, and hph-1-NOX4 double mutant animals prior to phenotyping for AAA and isolation of aortic endothelial cells for detection of miR-192-5p expression levels. Data are presented as Mean±SEM (n=4), ***p<0.001.

FIG. 3E shows the expression level of mmu-miR-192-5p in hph-1 and hph-1-NOX4 mice with and without Ang II infusion. Ang II was infused into hph-1, hph-1-NOX1, hph-1-NOX2, hph-1-p47phox, and hph-1-NOX4 double mutant animals prior to phenotyping for AAA and isolation of aortic endothelial cells for detection of miR-192-5p expression levels. Data are presented as Mean±SEM (n=6), **p<0.01, ***p<0.001.

FIG. 4A shows that mmu-miR-192-5p-specific inhibitors attenuated mmu-miR-192-5p expression in Ang II infused hph-1 mice. Mmu-miR-192-5p-specific inhibitors and negative controls were injected into Ang II infused hph-1 mice, and aortic endothelial cells were harvested to detect miR-192-5p expression levels. Data are presented as Mean±SEM (n=6), **p<0.01, ***p<0.001.

FIG. 4B shows that mmu-miR-192-5p specific inhibitors markedly restored DHFR mRNA in Ang II infused hph-1 mice. Mmu-miR-192-5p-specific inhibitors and negative controls were injected into Ang II infused hph-1 mice, and aortic endothelial cells were harvested to detect DHFR mRNA expression levels. Data are presented as Mean±SEM (n=4-6), **p<0.01, ***p<0.001.

FIG. 4C shows representative Western blots of endothelial DHFR protein expression with 3-actin serving as an internal control. Mmu-miR-192-5p-specific inhibitors and negative controls were injected into Ang II infused hph-1 mice, and aortic endothelial cells were harvested to detect DHFR protein expression levels.

FIG. 4D shows that mmu-miR-192-5p specific inhibitors markedly restored DHFR protein expression in Ang II infused hph-1 mice. Mmu-miR-192-5p-specific inhibitors and negative controls were injected into Ang II infused hph-1 mice, and aortic endothelial cells were harvested to detect DHFR protein expression levels. Data are presented as Mean±SEM (n=4), *p<0.05.

FIG. 5A shows that aortic production of superoxide detected by DHE imaging was markedly increased in Ang II infused hph-1 mice, which was substantially attenuated by in vivo treatment with mmu-miR-192-5p specific inhibitors. Mmu-miR-192-5p-specific inhibitors and negative controls were injected into Ang II infused hph-1 mice, and the aortas were freshly harvested for dihydroethidium (DHE) imaging analysis of superoxide production.

FIG. 5B shows quantitative analysis of fluorescent intensity of DHE images indicating the same result as in FIG. 5A. Mmu-miR-192-5p-specific inhibitors and negative controls were injected into Ang II infused hph-1 mice, and the aortas were freshly harvested for dihydroethidium (DHE) imaging analysis of superoxide production. Data are presented as Mean±SEM (n=4-5).**p<0.01, ***p<0.001.

FIG. 5C shows total superoxide production determined by ESR in the presence or absence of L-NAME (NOS inhibitor). There is a very modest eNOS uncoupling (L-NAME-inhibitable superoxide production) activity at baseline in hph-1 mice as previously published. The marked increase in eNOS uncoupling activity in Ang II-infused hph-1 mice was completely attenuated by in vivo treatment with mmu-miR-192-5p specific inhibitors. Mmu-miR-192-5p-specific inhibitors and negative controls were injected into Ang II infused hph-1 mice, and the aortas were freshly harvested for electron spin resonance (ESR) analyses of superoxide production. Data are presented as Mean±SEM (n=6-7). *p<0.05 vs. L-NAME(−) for all corresponding groups, #p<0.05 vs. Sham group without Ang II infusion, & p<0.05 vs. Ang II and Ang II+ negative control groups without L-NAME.

FIG. 5D shows that NO bioavailability determined by ESR was markedly decreased in Ang II infused hph-1 mice, which was significantly restored by in vivo treatment with mmu-miR-192-5p specific inhibitors. Mmu-miR-192-5p-specific inhibitors and negative controls were injected into Ang II infused hph-1 mice, and the aortas were freshly harvested for electron spin resonance (ESR) determination of NO bioavailability. Data are presented as Mean±SEM (n=7-9). *p<0.05, **p<0.01.

FIG. 6A shows the percentage of AAA in Ang II infused hph-1 mice treated with negative controls and mmu-miR-192-5p specific inhibitors. Mmu-miR-192-5p-specific inhibitors and negative controls were injected into Ang II infused hph-1 mice, prior to phenotyping of the mice for AAA formation. n=10-20, ***p<0.001.

FIG. 6B shows representative images of abdominal aortic expansion and inhibition defined by echocardiography in Ang II infused hph-1 mice and those treated with mmu-miR-192-5p specific inhibitors. Mmu-miR-192-5p-specific inhibitors and negative controls were injected into Ang II infused hph-1 mice, prior to phenotyping of the mice for AAA formation.

FIG. 6C shows time-dependent expansion of abdominal aortas defined by echocardiography was attenuated by in vivo treatment with miR-192-5p specific inhibitors in Ang II infused hph-1 mice. Mmu-miR-192-5p-specific inhibitors and negative controls were injected into Ang II infused hph-1 mice, prior to phenotyping of the mice for AAA formation by echocardiography. Data are presented as Mean±SEM (n=7) **p<0.01, ***p<0.001 vs. sham; #p<0.05 vs. Ang II; &p<0.05 vs. Ang II+ negative control.

FIG. 6D shows that postmortem inspection indicated that AAA formation was attenuated by in vivo treatment of mmu-miR-192-5p specific inhibitors in Ang II infused hph-1 mice. Mmu-miR-192-5p-specific inhibitors and negative controls were injected into Ang II infused hph-1 mice, prior to postmortem inspection phenotyping of the mice for AAA formation.

FIG. 6E shows that Ang II infusion into hph-1 mice induced a marked adventitial hypertrophy and intra-wall thrombosis, which was attenuated by in vivo treatment with mmu-miR-192-5p specific inhibitors. Mmu-miR-192-5p-specific inhibitors and negative controls were injected into Ang II infused hph-1 mice, prior to phenotyping of the mice for AAA formation by H&E imaging analysis.

FIG. 6F shows that Ang II infusion into hph-1 mice induced enlargement of external dimeters of the abdominal aortas, which was attenuated by in vivo treatment with mmu-miR-192-5p specific inhibitors. Mmu-miR-192-5p-specific inhibitors and negative controls were injected into Ang II infused hph-1 mice, prior to measurements of external diameters of the isolated abdominal aortas. Data are presented as Mean±SEM (n=10-11), *p<0.05,***p<0.001.

FIG. 6G shows VVG staining indicating significant degradation and flattening of elastic fibers in the aortic medial layers of Ang II infused hph-1 mice, which was restored by in vivo treatment with mmu-miR-192-5p specific inhibitors. Mmu-miR-192-5p-specific inhibitors and negative controls were injected into Ang II infused hph-1 mice, prior to phenotyping of the mice for medial layer matrix degradation.

FIG. 7 shows a schematic illustration of an intermediate role of miR-192-5p in the formation of AAA downstream of NOX. miR-192-5p is upregulated by H₂O₂ following NADPH oxidase (NOX) activation by Ang II infusion, leading to downregulation of dihydrofolate reductase (DHFR), uncoupling of eNOS that is accompanied by increased ROS production and reduced NO bioavailability, resulting in sustained oxidative stress, vascular remodeling and AAA formation.

### DETAILED DESCRIPTION OF THE INVENTION

### General

Oxidative stress plays an important role in the development of AAA, hypertension, ARDS and diseases associated with endothelial dysfunction. It was previously shown that an endothelial cell specific dihydrofolate reductase (DHFR) deficiency underlies Angiotensin II (Ang II) induced eNOS uncoupling and eNOS uncoupling-dependent formation of AAA in Ang II infused hyperphenylalaninemia (hph)-1 mice and apoE null mice, as well as development of hypertension in wildtype (WT) mice and DHFR knockout mice (Gao L et al, Hypertension 2012; Li Q et al, Redox Biology 2019). In hph-1 and apoE null mice, Ang II infusion augments eNOS uncoupling via downregulation of DHFR. Folic acid prevents progressive uncoupling of eNOS and vascular remodeling via restoration of DHFR function, resulting in completely normalized blood pressure in WT mice and abrogated AAA formation in Ang II-infused hph-1 mice and apoE null mice. Utilizing double knockout strategies, it was further shown that DHFR deficiency lies downstream of NOX isoforms 1, 2 or 4 in Ang II-infused hph-1 mice, in line with our previous findings that hydrogen peroxide (H₂O₂) produced by NOX induces DHFR deficiency. Mice knocked out of DHFR displayed phenotypes of more severe vascular remodeling, and exaggerated AAA and hypertension via mitochondrial dysfunction. Folic acid restoration of DHFR function to result in recoupling of eNOS in WT mice also substantially alleviates development of hypertension (Gao L et al, Hypertension 2012).

microRNAs (miRs) are small, endogenous, single-stranded non-coding RNA molecules, typically having 18-22 nucleotides. They bind to the 3′-untranslated regions (3′-UTRs) of specific messenger RNAs to induce their degradation or translational repression via an imperfect complement in animal cells or perfect complement in plant cells. miR-192-5p was reported to decrease DHFR protein expression via translational arrest. Yang et al. Oncotarget. 2015; 6:43712-30 reported that levels of miR-192-5p are reduced in medulloblastoma cells.

An intermediate role of miR-192-5p in NOX-dependent downregulation of DHFR, and subsequent formation of AAA was investigated as described herein. The expression level of miR-192-5p was substantially upregulated in the aortic aneurysmal tissues of human AAA patients (n=15 for both AAA patients and controls), H₂O₂ treated human aortic endothelial cells (HAECs,) and Ang II treated hph-1 mice, while it was decreased in double mutants of hph-1-NOX1, hph-1-NOX2, hph-1-p47phox, and hph-1-NOX4 mice. In vivo treatment with miR-192-5p specific inhibitors markedly restored DIFR mRNA and protein levels, decreased superoxide production, recoupled eNOS, restored NO bioavailability, and attenuated AAA formation. These results show that targeting miR-192-5p serves as a novel therapeutic approach for the treatment and/or prevention of AAA. In view of the protective effects of miR-192-5p inhibitors on DIFR function, miR-192-5p can be targeted as a novel treatment option for hypertension, as well as for ARDS and diseases associated with endothelial dysfunction. Of note, acute respiratory distress syndrome (ARDS) is another critical situation tightly linked to endothelial dysfunction in the lung that could be caused by endothelial DIFR deficiency, targeting of which by inhibition of miR-192-5p could prove to be highly beneficial.

In one aspect, provided herein are pharmaceutical compositions comprising a miRNA inhibitor (e.g., miR-192-5p inhibitor), which can be used to treat aneurysms, hypertension, ARDS and diseases associated with endothelial dysfunction.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification.

All of the above, and any other publications, patents and published patent applications referred to in this application are specifically incorporated by reference herein. In case of conflict, the present specification, including its specific definitions, will control.

The articles “a” and “an” are used herein to refer to one or to more than one (e.g., to at least one) of the grammatical object of the article. By way of example, “an element” means one element or more than one element.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

The term “binding” or “interacting” refers to an association, which may be a stable association, between two molecules, e.g., between a miRNA inhibitor and target miRNA, e.g., due to, for example, electrostatic, hydrophobic, ionic, and/or hydrogen-bond interactions under physiological conditions.

As used herein, two nucleic acid sequences “complement” one another or are “complementary” to one another if they base pair one another at each position.

As used herein, two nucleic acid sequences “correspond” to one another if they are both complementary to the same nucleic acid sequence.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“miRNA” refers to small, endogenous, single-stranded non-coding RNA molecule of 18-22 nucleotides that modulates gene expression via an effector nucleic acid-protein complex (e.g., microribonucleoprotein (RNP) or miRNA-induced silencing complex (RISC)). miRNA bind to the 3-untranslated regions (3-UTRs) of specific messenger RNAs to induce their degradation or translational repression. In some embodiments, the miRNA imperfectly complements the 3-UTR of the specific messenger RNA. In some embodiments, the miRNA perfectly complements the 3-UTR of the specific messenger RNA. MicroRNA molecules (“miRNAs”) are generally 21 to 22 nucleotides in length, though lengths of 17 and up to 25 nucleotides have been reported. The miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved by an enzyme called Dicer in animals. Dicer is ribonuclease III-like nuclease. The processed miRNA is typically a portion of the stem. The processed miRNA (also referred to as “mature miRNA”) becomes part of a large complex to down-regulate a target gene.

“miRNA inhibitor” refers to a nucleotide sequence that is a reverse complement of a mature miRNA (the target site). The miRNA inhibitor binds to the target site (e.g., miR-192-5p or those listed in Tables 1-4) and suppresses the function of the mature miRNA. In some embodiments, the miRNA inhibitor is chemically synthesized. In some embodiments, the miRNA inhibitor is chemically modified to prevent nucleic acid-protein complex-induced cleavage (e.g., RISC-induced cleavage) of miRNA, enhance binding affinity to the target site, and/or provide resistance to nucleolytic degradation of the miRNA inhibitor. In some embodiments, the miRNA inhibitors described herein are delivered to cells using a delivery vehicle, such as liposomes or cationic polymers. In some embodiments, the miRNA inhibitor described herein is chemically modified so as to not require the use of such delivery vehicles to mediate targeting of miRNA (e.g., targeting of miR-192-5p or those listed in Tables 1-4) in a cell. As used herein, an miRNA inhibitor includes any natural or artificial RNA transcripts that sequestrate miRNAs and decrease or eliminate their effects. Included herein are miRNA inhibitors that are identical to competing endogenous RNAs (ceRNAs). ceRNAs are natural and intracellular miRNA inhibitors that compete to bind to shared miRNA recognition elements (MREs) to decrease microRNA availability and relieve the repression of target RNAs.

As used herein, the terms “interfering nucleic acid,” “inhibiting nucleic acid” are used interchangeably. Interfering nucleic acids generally include a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence. Interfering RNA molecules include, but are not limited to, antisense molecules, siRNA molecules, asiRNA molecules, lasiRNA molecules, single-stranded siRNA molecules, miRNA molecules and shRNA molecules. Such an interfering nucleic acids can be designed to block or inhibit translation of mRNA or to inhibit natural premRNA splice processing, or induce degradation of targeted mRNAs, and may be said to be “directed to” or “targeted against” a target sequence with which it hybridizes. Interfering nucleic acids may include, for example, peptide nucleic acids (PNAs), locked nucleic acids (LNAs), 2′-O-Methyl oligonucleotides and RNA interference agents (siRNA agents). RNAi molecules generally act by forming a heteroduplex with the target molecule, which is selectively degraded or “knocked down,” hence inactivating the target RNA. Under some conditions, an interfering RNA molecule can also inactivate a target transcript by repressing transcript translation and/or inhibiting transcription of the transcript. An interfering nucleic acid is more generally said to be “targeted against” a biologically relevant target, such as a protein, when it is targeted against the nucleic acid of the target in the manner described above.

The terms “polynucleotide”, and “nucleic acid” are used interchangeably. They refer to a polymeric form of nucleotides, whether deoxyribonucleotides, ribonucleotides, or analogs thereof, in any combination and of any length. Polynucleotides may have any three dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as aneurysm, hypertension, acute respiratory distress syndrome (ARDS), a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art.

For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow-release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

The terms “identical” or “percent identity,” in the context of two or more nucleic acids, refer to two or more sequences or subsequences that are the same or have a specified percentage of nucleotides that are the same e.g., about 50% identity, preferably 55%, 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/ or the like).

The term “modulation” or “modulate,” when used in reference to a functional property or biological activity or process (e.g., enzyme activity or receptor binding), refers to the capacity to either up regulate (e.g., activate or stimulate), down regulate (e.g., inhibit or suppress) or otherwise change a quality of such property, activity, or process. In certain instances, such regulation may be contingent on the occurrence of a specific event, such as activation of a signal transduction pathway, and/or may be manifest only in particular cell types.

As used herein, “specific binding” refers to the ability of a miRNA inhibitor to bind to a predetermined miRNA target. Typically, a miRNA inhibitor specifically binds to its target with an affinity corresponding to a K_{D} of about 10⁻⁷ M or less, about 10⁻⁸ M or less, or about 10⁻⁹ M or less and binds to the target with a K_{D} that is significantly less (e.g., at least 2 fold less, at least 5 fold less, at least 10 fold less, at least 50 fold less, at least 100 fold less, at least 500 fold less, or at least 1000 fold less) than its affinity for binding to a non-specific and unrelated target (e.g., BSA, casein, or an unrelated cell, such as an HEK 293 cell or an E. coli cell).

The terms “polynucleotide” and “nucleic acid” are used herein interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans; antioxidants, such as ascorbic acid or glutathione; chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro-emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

miRNA Inhibitors

In certain aspects, provided herein are miRNA inhibitors (e.g., miR-192-5p inhibitor) that selectively binds to miRNA (e.g., miR-192-5p). In some aspects, provided herein are pharmaceutical compositions comprising such miRNA inhibitors, methods of using such miRNA inhibitors to prevent, inhibit, treat, or reduce aneurysms, hypertension, acute respiratory distress syndrome (ARDS), and/or other diseases associated with endothelial dysfunction, and methods of making such miRNA inhibitors.

Exemplary miRNA inhibitor sequences are provided in Table 1.

TABLE 1
Exemplary miRNA Inhibitor Sequences

| miRNA Inhibitor | SEQ ID | | |
|---|---|---|---|
| Name | NO. | Nucleotide Sequence | Target |
| hsa-miR-192-5p | 1 | GGCUGUCAAUUCAUAGG | hsa-miR-192-5p |
| inhibitor | | UCAG | |
| hsa-miR-192-3p | 2 | CUGUGACCUAUGGAAUU | hsa-miR-192-3p |
| inhibitor | | GGCAG | |
| hsa-miR-215-5p | 3 | GUCUGUCAAUUCAUAGG | hsa-miR-215-5p |
| inhibitor | | UCAU | |
| hsa-miR-215-3p | 4 | UAUUGGCCUAAAGAAAU | hsa-miR-215-3p |
| inhibitor | | GACAGA | |
| hsa-miR-33a-5p | 5 | UGCAAUGCAACUACAAUG | hsa-miR-33a-5p |
| inhibitor | | CAC | |
| hsa-miR-33a-3p | 6 | GUGAUGCACUGUGGAAA | hsa-miR-33a-3p |
| inhibitor | | CAUUG | |
| hsa-miR-33b-5p | 7 | GCAAUGCAACAGCAAUGC | hsa-miR-33b-5p |
| inhibitor | | AC | |
| hsa-miR-33b-3p | 8 | GGGCUGCACUGCCGAGGC | hsa-miR-33b-3p |
| inhibitor | | ACUG | |
| hsa-miR-155-5p | 9 | AACCCCUAUCACGAUUAG | hsa-miR-155-5p |
| inhibitor | | CAUUAA | |
| hsa-miR-155-3p | 10 | UGUUAAUGCUAAUAUGU | hsa-miR-155-3p |
| inhibitor | | AGGAG | |
| hsa-mir-192 | 11 | GCUGGCAUUGAGGCGAAC | hsa-mir-192 |
| precursor inhibitor | | AUACCUGUGACCUAUGGA | precursor |
| | | AUUGGCAGCCAGAGGGG | |
| | | AGACGAGAGCACUGGCUG | |
| | | UCAAUUCAUAGGUCAGA | |
| | | GCCCUGUGCACUCGGUCU | |
| | | CGGC | |
| hsa-mir-215 | 12 | UUGAAGUAGCACAGUCA | hsa-mir-215 |
| precursor inhibitor | | UACAGAAUAUUGGCCUA | precursor |
| | | AAGAAAUGACAGACAAA | |
| | | CUCAGCUAUAUUGUCUGU | |
| | | CAAUUCAUAGGUCAUUU | |
| | | UCCUGUAUACCAUUUCUG | |
| | | AAUGAU | |
| hsa-mir-192/215 | 13 | GAAGUAGCACAGUCAUAC | hsa-mir-192/215 |
| precursor inhibitor | | AGAAUAUUGGCCUAAAG | precursor |
| | | AAAUGACAGACAAACUCA | |
| | | GCUAUAUUGUCUGUCAA | |
| | | UUCAUAGGUCAUUUUCCU | |
| | | GUAUACCAUUUCUGAAU | |
| hsa-mir-192-P1 | 14 | CUGUGACCUAUGGAAUU | hsa-mir-192-P1 |
| precursor inhibitor | | GGCAGCCAGAGGGGAGAC | precursor |
| | | GAGAGCACUGGCUGUCAA | |
| | | UUCAUAGGUCAG | |
| hsa-mir-194-1 | 15 | UUGGUAACCAUCAAAAG | hsa-mir-194-1 |
| precursor inhibitor | | UAACAGCAUCUCCACUGG | precursor |
| | | AAAUUGGUACACAGUCCA | |
| | | CAUGGAGUUGCUGUUAC | |
| | | ACUUGAUAACACCAU | |
| hsa-mir-194-2 | 16 | CUGGCCCUCGCCCCAGAU | hsa-mir-194-2 |
| precursor inhibitor | | AACAGCAGCCCCACUGGA | precursor |
| | | ACCAGUGGGCACUUCCAC | |
| | | AUGGAGUUGCUGUUACA | |
| | | GGGGGCGGGAACCA | |
| hsa-mir-194-P2 | 17 | AAAGUAACAGCAUCUCCA | hsa-mir-194-P2 |
| precursor inhibitor | | CUGGAAAUUGGUACACA | precursor |
| | | GUCCACAUGGAGUUGCUG | |
| | | UUACA | |
| hsa-miR-194-5p | 18 | UCCACAUGGAGUUGCUGU | hsa-miR-194-5p |
| inhibitor | | UACA | |
| hsa-miR-194-3p | 19 | CAGAUAACAGCAGCCCCA | hsa-miR-194-3p |
| inhibitor | | CUGG | |

Exemplary miRNA sequences are provided in Table 2.

TABLE 2
Exemplary miRNA sequences

| | | Mirbase.org, | | |
|---|---|---|---|---|
| | | RNAcentral.org, or | | |
| | SEQ ID | MirGeneDB.org | Nucleotide | |
| miRNA Name | NO. | Accession | Sequence | Target |
| hsa-miR-192-5p | 20 | MIMAT0000222 | CUGACCUAUGAA | DHFR |
| | | | UUGACAGCC | |
| hsa-miR-192-3p | 21 | MIMAT0004543 | CUGCCAAUUCCA | |
| | | | UAGGUCACAG | |
| hsa-miR-215-5p | 22 | MIMAT0000272 | AUGACCUAUGAA | |
| | | | UUGACAGAC | |
| hsa-miR-215-3p | 23 | MIMAT0026476 | UCUGUCAUUUCU | |
| | | | UUAGGCCAAUA | |
| hsa-miR-33a-5p | 24 | MIMAT0000091 | GUGCAUUGUAGU | MMP9 |
| | | | UGCAUUGCA | |
| hsa-miR-33a-3p | 25 | MIMAT0004506 | CAAUGUUUCCAC | |
| | | | AGUGCAUCAC | |
| hsa-miR-33b-5p | 26 | MIMAT0003301 | GUGCAUUGCUGU | |
| | | | UGCAUUGC | |
| hsa-miR-33b-3p | 27 | MIMAT0004811 | CAGUGCCUCGGC | |
| | | | AGUGCAGCCC | |
| hsa-miR-155-5p | 28 | MIMAT0000646 | UUAAUGCUAAUC | CTLA4, |
| | | | GUGAUAGGGGUU | SMAD2 |
| hsa-miR-155-3p | 29 | MIMAT0004658 | CUCCUACAUAUU | CTLA4, |
| | | | AGCAUUAACA | SMAD2 |
| hsa-mir-192 | 30 | MI0000234 | GCCGAGACCGAG | |
| precursor | | | UGCACAGGGCUC | |
| | | | UGACCUAUGAAU | |
| | | | UGACAGCCAGUG | |
| | | | CUCUCGUCUCCC | |
| | | | CUCUGGCUGCCA | |
| | | | AUUCCAUAGGUC | |
| | | | ACAGGUAUGUUC | |
| | | | GCCUCAAUGCCA | |
| | | | GC | |
| hsa-mir-215 | 31 | MI0000291 | AUCAUUCAGAAA | |
| precursor | | | UGGUAUACAGGA | |
| | | | AAAUGACCUAUG | |
| | | | AAUUGACAGACA | |
| | | | AUAUAGCUGAGU | |
| | | | UUGUCUGUCAUU | |
| | | | UCUUUAGGCCAA | |
| | | | UAUUCUGUAUGA | |
| | | | CUGUGCUACUUC | |
| | | | AA | |
| hsa-mir-192/215 | 32 | URS0000651765_ | AUUCAGAAAUGG | |
| precursor | | 9606 | UAUACAGGAAAA | |
| | | | UGACCUAUGAAU | |
| | | | UGACAGACAAUA | |
| | | | UAGCUGAGUUUG | |
| | | | UCUGUCAUUUCU | |
| | | | UUAGGCCAAUAU | |
| | | | UCUGUAUGACUG | |
| | | | UGCUACUUC | |
| hsa-mir-192-P1 | 33 | Hsa-Mir-192-P1 | CUGACCUAUGAA | |
| precursor | | | UUGACAGCCAGU | |
| | | | GCUCUCGUCUCC | |
| | | | CCUCUGGCUGCC | |
| | | | AAUUCCAUAGGU | |
| | | | CACAG | |
| hsa-mir-194-1 | 34 | MI0000488 | AUGGUGUUAUCA | |
| precursor | | | AGUGUAACAGCA | |
| | | | ACUCCAUGUGGA | |
| | | | CUGUGUACCAAU | |
| | | | UUCCAGUGGAGA | |
| | | | UGCUGUUACUUU | |
| | | | UGAUGGUUACCA | |
| | | | A | |
| hsa-mir-194-2 | 35 | MI0000732 | UGGUUCCCGCCC | |
| precursor | | | CCUGUAACAGCA | |
| | | | ACUCCAUGUGGA | |
| | | | AGUGCCCACUGG | |
| | | | UUCCAGUGGGGC | |
| | | | UGCUGUUAUCUG | |
| | | | GGGCGAGGGCCA | |
| | | | G | |
| hsa-mir-194-P2 | 36 | Hsa-Mir-194-P2 | UGUAACAGCAAC | |
| precursor | | | UCCAUGUGGACU | |
| | | | GUGUACCAAUUU | |
| | | | CCAGUGGAGAUG | |
| | | | CUGUUACUUU | |
| hsa-miR-194-5p | 37 | MIMAT0000460 | UGUAACAGCAAC | |
| | | | UCCAUGUGGA | |
| hsa-miR-194-3p | 38 | MIMAT0004671 | CCAGUGGGGCUG | |
| | | | CUGUUAUCUG | |

DHFR is predicted to be the putative target of miR-192-5p by TargetScan as shown below:

Position 648-554 of DHFR 3′ UTR
5′ . . . AAGCAGUGUAUUUGCUAGGUCAU . . .
|||||||
3′ CCGACAGUUAAGUAUCCAGUC
hsa-mIR-192-5p

miR-192-5p is conserved between different species (Table 3). miR-192-5p is highly conserved between different species, especially in the seed region (bold) that targets dihydrofolate reductase (DIR). All of the sequences of miR-192-5p from different species were acquired from the miRBase (http://www.mirbase.org/).

TABLE 3

| | miRBase | |
|---|---|---|
| Name | Accession | Sequence |
| hsa-miR-192- | MIMAT0000222 | CUGACCUAUGAAUUGACAGCC |
| 5p | | |
| mmu-miR-192- | MIMAT0000517 | CUGACCUAUGAAUUGACAGCC |
| 5p | | |
| rno-miR-192- | MIMAT0000867 | CUGACCUAUGAAUUGACAGCC |
| 5p | | |
| fru-miR-192 | MIMAT0002941 | AUGACCUAUGAAUUGACAGCC |
| tch-miR-192- | MIMAT0036616 | CUGACCUAUGAAUUGACAGCC |
| 5p | | |
| pma-miR-192- | MIMAT0019497 | UUGACCUAUGAAUUGACAGCCG |
| 5p | | |
| oan-miR-192- | MIMAT0007016 | CUGACCUAUGAAUUGACAGCC |
| 5p | | |
| mml-miR-192- | MIMAT0006224 | CUGACCUAUGAAUUGACAGCC |
| 5p | | |
| cpi-miR-192- | MIMAT0037807 | AUGACCUAUGAAUUGACAGCC |
| 5p | | |
| gmo-miR-192- | MIMAT0044277 | AUGACCUAUGAAUUGACAGCCA |
| 5p | | |
| nle-miR-192 | MIMAT0049384 | CUGACCUAUGAAUUGACAGCC |
| eca-miR-192 | MIMAT0013049 | CUGACCUAUGAAUUGACAGCC |
| ami-miR-192- | MIMAT0038206 | UGACCUAUGGAUUGACAGCCAG |
| 5p | | |
| bta-miR-192 | MIMAT0003820 | CUGACCUAUGAAUUGACAGCCAG |
| abu-miR-192 | MIMAT0042042 | AUGACCUAUGAAUUGACAGCC |

The gene IDs and NCBI reference sequences of the miRNAs in Table 2 are shown below in Table 4.

TABLE 4

| Name | Gene ID | NCBI Reference Sequence |
|---|---|---|
| hsa-miR- | HGNC: 31562 | GCCGAGACCGAGTGCACAGGGCTCT |
| 192 | NCBI Entrez Gene: 406967 | GACCTATGAATTGACAGCCAGTGCT |
| | Ensembl: ENSG00000283926 | CTCGTCTCCCCTCTGGCTGC |
| | OMIM®: 610939 | |
| | miRBase: hsa-mir-192 | |
| | GeneCards ID: GC11M064891 | |
| hsa-mi- | RNAcentral.org ID: | GGCTGTCAATTCATAGGTCAG |
| 192-5p | URS0000155642_9606 | (RNAcentral.org) |
| hsa-miR- | HGNC: 31592 | ATCATTCAGAAATGGTATACAGGAA |
| 215 | NCBI Entrez Gene: 406997 | AATGACCTATGAATTGACAGACAAT |
| | Ensembl: ENSG00000207590 | ATAGCTGAGTTTGTCTGTCASTTTCT |
| | OMIM®: 610943 | TTAGGCCAATATTCTGTATGACTGTG |
| | miRBase: hsa-mir-215 | CTACTTCAA |
| | GeneCards ID: GC01M220117 | |
| hsa-miR- | HGNC: 31634 | CTGTGGTGCATTGTAGTTGCATTGCA |
| 33a | NCBI Entrez Gene: 407039 | TGTTCTGGTGGTACCCATGCAATGTT |
| | Ensembl: ENSG00000207932 | TCCACAGTGCATCACAG |
| | OMIM®: 612156 | |
| | miRBase: hsa-mir-33a | |
| | GeneCards ID: GC22P041900 | |
| hsa-miR- | HGNC: 32791 | GCGGGCGGCCCCGCGGTGCATTGCT |
| 33b | NCBI Entrez Gene: 693120 | GTTGCATTGCACGTGTGTGAGGCGG |
| | Ensembl: ENSG00000207839 | GTGCAGTGCCTCGGCAGTGCAGCCC |
| | OMIM®: 613486 | GGAGCCGGCCCCTGGCACCAC |
| | miRBase: hsa-mir-33b | |
| | GeneCards ID: GC17M017813 | |
| hsa-miR- | HGNC: 31542 | CTGTTAATGCTAATCGTGATAGGGG |
| 155 | NCBI Entrez Gene: 406947 | TTTTTGCCTCCAACTGACTCCTACAT |
| | Ensembl: ENSG00000283904 | ATTAGCATTAACAG |
| | OMIM®: 609337 | |
| | miRBase: hsa-mir-155 | |
| | GeneCards ID: GC21P025573 | |
| hsa-miR- | HGNC: 31564 | ATGGTGTTATCAAGTGTAACAGCAA |
| 194-1 | NCBI Entrez Gene: 406969 | CTCCATGTGGACTGTGTACCAATTTC |
| | Ensembl: ENSG00000207624 | CAGTGGAGATGCTGTTACTTTTGAT |
| | OMIM®: 610940 | GGTTACCAA |
| | miRBase: hsa-mir-194-1 | |
| | GeneCards ID: GC01M220118 | |
| hsa-miR- | HGNC: 31565 | TGGTTCCCGCCCCCTGTAACAGCAA |
| 194-2 | NCBI Entrez Gene: 406970 | CTCCATGTGGAAGTGCCCACTGGTT |
| | Ensembl: ENSG00000284155 | CCAGTGGGGCTGCTGTTATC |
| | OMIM®: 610941 | TGGGGCGAGGGCCAG |
| | miRBase: hsa-mir-194-2 | |
| | GeneCards ID: GC11M065575 | |

In some embodiments, the miRNA inhibitor comprises a nucleic acid that is at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, least 81%, least 82%, least 83%, least 84%, least 85%, least 86%, least 87%, least 88%, least 89%, at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, or at least 99.9% sequence identity) to the nucleic acid sequence of any one of SEQ ID NOs: 1-19. In some embodiments, the miRNA inhibitor comprises a nucleic acid that is any one of SEQ ID NOs: 1-19.

In some embodiments, the nucleic acid is complementary to any one of SEQ ID NOs: 20-38.

In some embodiments, the miRNA inhibitor binds to a miRNA comprising a nucleic acid that is at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, least 81%, least 82%, least 83%, least 84%, least 85%, least 86%, least 87%, least 88%, least 89%, at least 90%, at least 91%, at least 92%, at least 93%, at least 94%, at least 95%, at least 96%, at least 97%, at least 98%, at least 99% sequence identity, or at least 100% sequence identity (e.g., at least 99.5% sequence identity, at least 99.6% sequence identity, at least 99.7% sequence identity, at least 99.8% sequence identity, at least 99.9% sequence identity, or at least 100% sequence identity) to the nucleic acid sequence of any one of SEQ ID NOs. 20-38. In some embodiments, the miRNA inhibitor binds to a miRNA comprising a nucleic acid that is any one of SEQ ID NOs. 20-38.

In some embodiments, the miRNA inhibitors are for example at least 5, 10, 20, 25, 30, 35, 40, 45, 50, 55, 60, 65, 70, 75, 80, 85, 90, 95, 100, 105, 110, 115, or 120 nucleotides in length. In some embodiments, the miRNA inhibitors are no more than 120, 115, 110, 100, 95, 90, 85, 80, 75, 70, 65, 60, 55, 50, 45, 40, 35, 30, 25, 20, 10, or 5 nucleotides in length. In some embodiments, the miRNA inhibitors are at least 18 nucleotides in length. In some embodiments, the miRNA inhibitors are no more than 22 nucleotides in length. A miRNA inhibitor disclosed herein may comprise at least 1, 2, 3, 4, 5, 6, 7, 8, 9, 10, 11, 12, 13, 14, 15, 16, 17, 18, 19, or 20 substitutions. A miRNA inhibitor disclosed herein may have no more than 40, 41, 42, 43, 44, 45, 46, 47, 48, 49, 50 substitutions as compared to any one of SEQ ID NOs:1-19.

In other embodiments of the invention, there are synthetic nucleic acids that are miRNA inhibitors. A miRNA inhibitor is between about 7 to 35 nucleotides (e.g., 17 to 25 nucleotides) in length and comprises a 5′ to 3′ sequence that is at least 90% complementary to the 5′ to 3′ sequence of a mature miRNA. In certain embodiments, a miRNA inhibitor molecule is 17, 18, 19, 20, 21, 22, 23, 24, or 25 nucleotides in length, or any range derivable therein. Moreover, an miRNA inhibitor has a sequence (from 5′ to 3′) that is or is at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA. One of skill in the art could use that portion of the longer probe sequence that is has at least partial complementarity to at least a portion of a sequence of a mature miRNA as the sequence for a miRNA inhibitor. Table 2 indicates mature sequences of miRNAs. Moreover, that portion of the probe sequence can be altered so that it is, for example, 90% complementary to the sequence of a mature miRNA.

In certain embodiments, a miRNA inhibitor competes with a miRNA inhibitor (e.g., an endogenous miRNA inhibitor) as described herein for binding to a miRNA comprising a nucleic acid that is any one of SEQ ID NOs: 20-38.

In certain embodiments, a vector comprises the miRNA inhibitor as described herein.

In certain embodiments, the miRNA inhibitors provided herein comprise one or more chemical modifications, wherein the modification facilitates the penetration of a cellular membrane in the absence of a delivery vehicle. Any chemical modification of the miRNA inhibitor that sustains the same functional structure will bind with its intended target.

In certain embodiments, examples of any of the chemical modifications include a 2′-O-methylated nucleoside (2′OMe), a 2′-fluoro oligonucleotide (2′F), a 2′-O-methoxyethyl oligonucleotide (2′MOE), a N6-methyladenosine (m⁶A), a phosphorodiamidate morpholino oligonucleotide (PMO), a peptide nucleic acid (PNA), a phosphorothioate bond (PS), a locked nucleic acid (LNA), a non-nucleotide N,N-diethyl-4-(4-nitronaphthalen-1-ylazo)-phenylamine (ZEN), a hydrophobic moiety, a naphthyl modifier, or a cholesterol moiety.

N,N-diethyl-4-(4-nitronaphthalen-1-ylazo)-phenylamine (ZEN) is a compound that increases binding affinity to a target oligonucleotide and blocks exonuclease degradation when placed at or near each end of an oligonucleotide.

In certain embodiments, the chemical modification of the miRNA is produced by methylation. In some embodiments, methylation of the miRNA inhibitor is mediated by HEN1 methyltransferase. HEN1 methyltransferase methylates the terminal ribose in short double-stranded RNAs (e.g., 3′ end labeling of a miRNA inhibitor). In some embodiments, methylation of the miRNA inhibitor by HEN1 methyltransferase produces a 2′-O-methylated nucleoside (2′OMe) chemical modification.

In certain embodiments, the miRNA inhibitor is not cytotoxic. The miRNA inhibitors described herein can employ a variety of oligonucleotide chemistries. Examples of oligonucleotide chemistries include, without limitation, peptide nucleic acid (PNA), linked nucleic acid (LNA), phosphorothioate, 2′O-Me-modified oligonucleotides, and morpholino chemistries, including combinations of any of the foregoing. In general, PNA and LNA chemistries can utilize shorter targeting sequences because of their relatively high target binding strength relative to 2′O-Me oligonucleotides. Phosphorothioate and 2′O-Me-modified chemistries are often combined to generate 2′OMe-modified oligonucleotides having a phosphorothioate backbone. See, e.g., PCT Publication Nos. WO/2013/112053 and WO/2009/008725, each of which is hereby incorporated by reference in its entirety. Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligonucleotides obeying Watson Crick base-pairing rules, and mimic DNA in terms of base pair recognition. The backbone of PNAs is formed by peptide bonds rather than phosphodiester bonds, making them wellsuited for antisense applications (see structure below). The backbone is uncharged, resulting in PNA/DNA or PNA/RNA duplexes that exhibit greater than normal thermal stability. PNAs are not recognized by nucleases or proteases. Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration and triplex formation with homopurine DNA. PANAGENE.TM. has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling and capping. PNAs can be produced synthetically using any technique known in the art. See, e.g., U.S. Pat. Nos. 6,969,766, 7,211,668, 7,022,851, 7,125,994, 7,145,006 and 7,179,896. See also U.S. Pat. Nos. 5,539,082; 5,714,331; and 5,719,262 for the preparation of PNAs. Further teaching of PNA compounds can be found in Nielsen et al., Science, 254:1497-1500, 1991. Each of the foregoing is incorporated by reference in its entirety. Interfering nucleic acids may also contain “locked nucleic acid” subunits (LNAs). “LNAs” are a member of a class of modifications called bridged nucleic acid (BNA). BNA is characterized by a covalent linkage that locks the conformation of the ribose ring in a C3-endo (northern) sugar pucker. For LNA, the bridge is composed of a methylene between the 2′-O and the 4′-C positions. LNA enhances backbone preorganization and base stacking to increase hybridization and thermal stability.

The structures of LNAs can be found, for example, in Wengel, et al., Chemical Communications (1998) 455; Tetrahedron (1998) 54:3607, and Accounts of Chem. Research (1999) 32:301); Obika, et al., Tetrahedron Letters (1997) 38:8735; (1998) 39:5401, and Bioorganic Medicinal Chemistry (2008) 16:9230. Compounds provided herein may incorporate one or more LNAs; in some cases, the compounds may be entirely composed of LNAs. Methods for the synthesis of individual LNA nucleoside subunits and their incorporation into oligonucleotides are described, for example, in U.S. Pat. Nos. 7,572,582, 7,569,575, 7,084,125, 7,060,809, 7,053,207, 7,034,133, 6,794,499, and 6,670,461, each of which is incorporated by reference in its entirety. Typical intersubunit linkers include phosphodiester and phosphorothioate moieties; alternatively, nonphosphorous containing linkers may be employed. One embodiment is an LNA-containing compound where each LNA subunit is separated by a DNA subunit. Certain compounds are composed of alternating LNA and DNA subunits where the intersubunit linker is phosphorothioate.

In certain embodiments, the miRNA inhibitor is linked to a cholesterol moiety. In some embodiments, the cholesterol moiety is attached to the 3′ terminus of the sense strand. In some embodiments, the cholesterol moiety is attached to the 3′ terminus of the antisense strand. In some embodiments, the cholesterol moiety is attached to the 5′ terminus of the sense strand. In some embodiments, the cholesterol moiety is attached to the 5′ terminus of the antisense strand.

In some embodiments, the miRNA inhibitor comprises a 2′-O-methylated nucleoside. 2′-O-methylated nucleosides carry a methyl group at the 2′-OH residue of the ribose molecule. 2′-O-Me-RNAs show the same (or similar) behavior as RNA, but are protected against nuclease degradation. 2′-O-Me-RNAs can also be combined with phosphothioate oligonucleotides (PTOs) for further stabilization. 2′-O-Me-RNAs (phosphodiester or phosphothioate) can be synthesized according to routine techniques in the art (see, e.g., Yoo et al., Nucleic Acids Res. 32:2008-16, 2004, which is hereby incorporated by reference). In some embodiments, the 2′-O-methyl nucleoside is positioned at the 3′ terminus of the sense strand. In some embodiments, 3′ terminal region of the sense strand comprises a plurality of 2′-O-methylated nucleosides (e.g., 2, 3, 4, 5 or 6 2′-O-methylated nucleosides within 6 nucleosides of the 3′ terminus). In some embodiments, the 2′-O-methyl nucleoside is positioned at the 3′ terminus of the antisense strand. In some embodiments, 3′ terminal region of the antisense strand comprises a plurality of 2′-O-methylated nucleosides (e.g., 2, 3, 4, 5 or 6 2′-O-methylated nucleosides within 6 nucleosides of the 3′ terminus). In some embodiments, both the 3′ terminal region of the sense strand and the 3′ terminal region of the antisense strand comprise a plurality of 2′-O-methylated nucleosides. In some embodiments, the sense strand comprises 2′-O-methylated nucleosides that alternate with unmodified nucleosides. In some embodiments, the sense strand comprises a contiguous sequence of 2, 3, 4, 5, 6, 7 or 8 2′-O-methylated nucleosides that alternate with unmodified nucleosides. In some embodiments, the anti-sense strand comprises 2′-O-methylated nucleosides that alternate with unmodified nucleosides. In some embodiments, the antisense strand comprises a contiguous sequence of 2, 3, 4, 5, 6, 7 or 8 2′-O-methylated nucleosides that alternate with unmodified nucleosides.

In some embodiments, the miRNA inhibitor comprises a phosphorothioate bond. a non-nucleotide N,N-diethyl-4-(4-nitronaphthalen-1-ylazo)-phenylamine (ZEN), “Phosphorothioates” (or S-oligos) are a variant of normal DNA in which one of the nonbridging oxygens is replaced by a sulfur. The sulfurization of the internucleotide bond reduces the action of endo—and exonucleases including 5′ to 3′ and 3′ to 5′ DNA POL 1 exonuclease, nucleases SI and P1, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1,2-benzodithiol-3-one 1,1-dioxide (BDTD) (see, e.g., Iyer et al., J. Org. Chem. 55, 20 4693-4699, 1990). The latter methods avoid the problem of elemental sulfur's insolubility in most organic solvents and the toxicity of carbon disulfide. The TETD and BDTD methods also yield higher purity phosphorothioates. In some embodiments, at least 25%, 30%, 35%, 40%, 45%, 50%, 55%, 60%, 65%, 70%, 75%, 80%, 85%, 90% or 95% of the bonds between the ribonucleotides in the sense strand of the miRNA inhibitor are phosphorothioate bonds. In some embodiments, all of the bonds between the ribonucleotides in the sense strand of the miRNA inhibitor are phosphorothioate bonds. In some embodiments, at least 25%, 30%, 35%, 40%, 45%, 50%, 55%, 60%, 65%, 70%, 75%, 80%, 85%, 90% or 95% of the bonds between the ribonucleotides in the antisense strand of the miRNA inhibitor are phosphorothioate bonds. In some embodiments, all of the bonds between the ribonucleotides in the antisense strand of the miRNA inhibitor are phosphorothioate bonds.

The miRNA inhibitors described herein may be contacted with a cell or administered to an organism (e.g., a human). Alternatively, constructs and/or vectors encoding the miRNA inhibitors may be contacted with or introduced into a cell or organism. In certain embodiments, a viral, retroviral or lentiviral vector is used. The miRNA inhibitors described herein can be prepared by any appropriate method known in the art. For example, in some embodiments, the miRNA inhibitors described herein are prepared by chemical synthesis or in vitro transcription.

In some embodiments, the cell is contacted with the miRNA inhibitor in the presence of a delivery vehicle (e.g., a liposome, cationic polymer, cell penetrating peptide (CPPs), protein transduction domain (PTDs), antibody and/or aptamer).

In the present methods, a miRNA inhibitor described herein can be administered to the subject, for example, as nucleic acid without a delivery vehicle, in combination with a delivery reagent, and/or as a nucleic acid comprising sequences that express the miRNA inhibitor described herein. In some embodiments, any nucleic acid delivery method known in the art can be used in the methods described herein. Suitable delivery reagents include, but are not limited to, e.g., the Mirus Transit TKO lipophilic reagent; lipofectin; lipofectamine; cellfectin; polycations (e.g., polylysine), atelocollagen, nanoplexes and liposomes. The use of atelocollagen as a delivery vehicle for nucleic acid molecules is described in Minakuchi et al. Nucleic Acids Res., 32(13):e109 (2004); Hanai et al. Ann NY Acad Sci., 1082:9-17 (2006); and Kawata et al. Mol Cancer Ther., 7(9):2904-12 (2008); each of which is incorporated herein in their entirety. Exemplary interfering nucleic acid delivery systems are provided in U.S. Pat. Nos. 8,283,461, 8,313,772, 8,501,930. 8,426,554, 8,268,798 and 8,324,366, each of which is 5 hereby incorporated by reference in its entirety.

In some embodiments of the methods described herein, liposomes are used to deliver a miRNA inhibitor described herein to a subject. Liposomes suitable for use in the methods described herein can be formed from standard vesicle-forming lipids, which generally include neutral or negatively charged phospholipids and a sterol, such as 10 cholesterol. The selection of lipids is generally guided by consideration of factors such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by 15 reference. The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure. 20 Opsonization-inhibiting moieties for use in preparing the liposomes described herein are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of 25 membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is herein incorporated by reference. In some embodiments, opsonization inhibiting moieties suitable for modifying 30 liposomes are water-soluble polymers with a number-average molecular weight from about 500 to about 40,000 daltons, or from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as OPH-00301-23-polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives 5 thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic 10 acid, carrageenan; aminated polysaccharides or oligosaccharides (linear or branched); or carboxylated polysaccharides or oligosaccharides, e.g., reacted with derivatives of carbonic acids with resultant linking of carboxylic groups. In some embodiments, the opsonizationinhibiting moiety is a PEG, PPG, or derivatives thereof. Liposomes modified with PEG or PEG-derivatives are sometimes called “PEGylated liposomes.”

In certain embodiments, the miRNA inhibitors comprise a terminal modification. In some embodiments, the miRNA inhibitors are chemically modified with polyethylene glycol (PEG) (e.g., 0.5-40 kDa) (e.g., attached to the 5′ end of the miRNA inhibitor). In some embodiments, the miRNA inhibitors comprise a 5′ end cap (e.g., an inverted thymidine, biotin, albumin, chitin, chitosan, cellulose, terminal amine, alkyne, azide, thiol, maleimide, NHS). In certain embodiments, the miRNA inhibitors comprise a 3′ end cap (e.g., an inverted thymidine, biotin, albumin, chitin, chitosan, cellulose, terminal amine, alkyne, azide, thiol, maleimide, NHS).

In certain embodiments, the miRNA inhibitors provided herein comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, 35, 36, 37, 38, 39, 40, 41, 42, 43, 44, 45, 46, 47, 48, 49, 50, 51, 52, 53, or 54) modified sugars. In some embodiments, the miRNA inhibitors comprise one or more 2′ sugar substitutions (e.g., a 2′-fluoro, a 2′-amino, or a 2′-O-methyl substitution). In certain embodiments, the miRNA inhibitors comprise locked nucleic acid (LNA), unlocked nucleic acid (UNA) and/or 2′deoxy-2′fluoro-D-arabinonucleic acid (2′-F ANA) sugars in their backbone.

In certain embodiments, the miRNA inhibitors comprise one or more (e.g., at least 1, 2,3,4, 5,6,7, 8,9, 10 11, 12, 13, 14, 15, 16, 17, 18, 19, 20, 21, 22, 23, 24, 25, 26, 27, 28, 29, 30 31, 32, 33, 34, 35, 36, 37, 38, 39, 40, 41, 42, 43, 44, 45, 46, 47, 48, 49, 50, 51, 52, 53, or 54) methylphosphonate internucleotide bonds, phosphorothioate internucleotide bonds, and/or phosphorodithioate internucleotide bonds. In certain embodiments, the miRNA inhibitors comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, 35, 36, 37, 38, 39, 40, 41, 42, 43, 44, 45, 46, 47, 48, 49, 50, 51, 52, 53, or 54) triazole internucleotide bonds. In certain embodiments, the miRNA inhibitors are modified with a cholesterol or a dialkyl lipid (e.g., on their 5′ end).

In some embodiments, the miRNA inhibitors comprise one or more modified nitrogenous bases (e.g., (benzylcarboxyamide)-deoxyuridine (BzdU), 5-methyl cytosine) or a nitrogenous base that comprise a functional group (e.g., a naphtyl, triptamino, isobutyl, or alkyne (dibenzocyclooctyne, azide, maleimide).

In some embodiments, the miRNA inhibitors described herein are labeled with and/or comprises a detectable label. In some embodiments, any detectable label can be used. Examples of detectable labels include, but are not limited to, fluorescent moieties, radioactive moieties, paramagnetic moieties, luminescent moieties and/or colorimetric moieties. In some embodiments, the miRNA inhibitors described herein are linked to, comprise and/or are bound by a fluorescent moiety. Examples of fluorescent moieties include, but are not limited to, Allophycocyanin (APC), Fluorescein, Fluorescein isothiocyanate (FITC), Phycoerythrin (PE), Cy3 dye, Cy5 dye, Peridinin-chlorophyll protein complex, Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 568, Alexa Fluor 594, Alexa Fluor 633, Alexa Fluor 635, Alexa Fluor 647, Alexa Fluor 660, Alexa Fluor 680, Alexa Fluor 700, Alexa Fluor 750, Alexa Fluor 790, EGFP, mPlum, mCherry, mOrange, mKO, EYFP, mCitrine, Venus, YPet, Emerald, Cerulean and CyPet.

miRNA inhibitors may be synthesized by methods that are well known to the skilled person. For example, miRNA inhibitors may be chemically synthesized, e.g. on a solid support. Solid phase synthesis may use phosphoramidite chemistry. Briefly, a solid supported nucleotide is detritylated, then coupled with a suitably activated nucleoside phosphoramidite to form a phosphite triester linkage. Capping may then occur, followed by oxidation of the phosphite triester with an oxidant, e.g., iodine. The cycle may then be repeated to assemble the miRNA inhibitors.

In certain aspects, provided herein are methods of making a miRNA inhibitor comprising synthesizing a nucleic acid molecule comprising a nucleic acid sequence that is at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity, or at least 100% sequence identity (e.g., at least 99.5% sequence identity, at least 99.6% sequence identity, at least 99.7% sequence identity, at least 99.8% sequence identity, at least 99.9% sequence identity, or at least 100% sequence identity) identical to the nucleic acid sequence of any one of SEQ ID NOs: 1-19. In some embodiments, the methods comprise synthesizing a nucleic acid molecule of any one of SEQ ID NOs: 1-19.

In certain aspects, the methods provided herein comprise preventing, inhibiting, treating, or reducing aneurysms, comprising administering a miRNA inhibitor as described herein to a subject in need thereof.

In certain aspects, the methods provided herein comprise preventing, inhibiting, treating, or reducing aneurysms, comprising administering a vector as described herein to a subject in need thereof.

In certain aspects, the methods provided herein comprise preventing, inhibiting, treating, or aneurysms, comprising administering a pharmaceutical composition as described herein to a subject in need thereof. In some embodiments, pharmaceutical composition is administered subcutaneously. In some embodiments, the pharmaceutical composition is administered parenterally.

In some embodiments, the aneurysm is abdominal aortic aneurysm, cerebral aneurysm, or thoracic aortic aneurysm. In some embodiments, the disease conditions is hypertension, ARDS, or any other pathological disorders associated with endothelial dysfunction.

In some embodiments, the method further comprise conjointly administering to the subject an additional therapeutic agent. In some embodiments, the additional therapeutic agent is a folate compound and/or a calcium channel blocker.

In certain aspects, the methods provided herein comprise reversing vascular remodeling, comprising administering the pharmaceutical composition as described herein to a subject in need thereof, wherein vascular remodeling is characterized by inflammation, matrix degradation, adventitial hypertrophy, medial elastin degradation and flattening, and/or formation of intra-lumen thrombi.

In certain aspects, the methods provided herein comprise modulating dihydrofolate reductase (DHFR), cytotoxic T-lymphocyte-associated protein (CTLA4), matrix metallopeptidase 9 (MMP-9) and/or SMAD Family 2 (SMAD2) mRNA expression and protein levels, comprising administering the pharmaceutical composition as described herein to a subject in need thereof.

In certain aspects, the methods provided herein comprise decreasing reactive oxygen species production, comprising administering the pharmaceutical composition as described herein to a subject in need thereof. Reactive oxygen species (ROS) are unstable molecules containing oxygen as a byproduct of the metabolism of oxygen, sometime containing a nitrogen as well being referred to reactive nitrogen species (RNS) at the same time (e.g. nitric oxide, peroxynitrite). ROS include superoxide, hydrogen peroxide, nitric oxide, peroxynitrite, hydrochlorous acid (HOCI), and hydroxyl radical. In some embodiments, reactive oxygen species production is measured by flow cytometry or fluorescent microscopy by staining for reactive oxygen species (e.g., superoxide, hydrogen peroxide, nitric oxide, peroxynitrite, hydrochlorous acid, or hydroxyl radical) with a fluorescent probe (e.g., dihydroethidium or 2′,7′-dichlorodihydrofluorescein diacetate (H2DCFDA). For example, reactive oxygen species production may be measured by dihydroethidium (DHE) staining for superoxide detection.

In certain aspects, the methods provided herein comprise restoring endothelial nitric oxide synthase (eNOS) coupling activity, comprising administering the pharmaceutical composition as described herein to a subject in need thereof. As used herein, “restoring endothelial nitric oxide synthase (eNOS) coupling activity” refers to increasing eNOS coupling activity relative to a previous measurement or a reference control sample. In certain embodiments, eNOS coupling/uncoupling activity is measured by electron spin resonance (ESR) spectroscopy in the presence of absence of L-NAME (an NOS inhibitor). ESR spectroscopy measures superoxide levels. If eNOS coupling activity is restored in the presence of L-NAME, the measure of superoxide levels will increase. If eNOS coupling activity is not restored in the presence of L-NAME, the measure of superoxide levels will remain decreased indicating superoxide production from eNOS/eNOS-derived superoxide production.

In certain aspects, the methods provided herein comprise preserving nitric oxide (NO) bioavailability, comprising administering the pharmaceutical composition as described herein to a subject in need thereof. Nitric oxide (NO) is a multifunctional signaling molecule involved in the maintenance of cardiovascular homeostasis. NO bioavailability indicates the actual endothelial NO molecules available to the function of the organ or cell systems, and its decrease is consequent to oxidative stress resulting in endothelial dysfunction. As used herein, “preserving NO bioavailability” refers to maintaining or increasing NO levels relative to a previous measurement or a reference control sample. In certain embodiments, NO availability is measured by electron spin resonance (ESR) spectroscopy. If NO bioavailability is preserved/restored, the measure of NO levels will increase. If NO bioavailability is not preserved, the measure of NO levels will remain decreased.

In certain aspects, the methods provided herein comprise method of decreasing miRNA expression, comprising administering the pharmaceutical composition as described herein to a subject in need thereof, wherein the miRNA comprises a nucleic acid that is any one of SEQ ID NOs: 20-38.

In certain aspects, the methods provided herein comprise method of making a miRNA inhibitor as described herein, comprising synthesizing a nucleic acid molecule.

### Method of Treating Disease

In certain aspects, the compositions and methods provided herein are useful for the treatment or prevention of a disease, disorder, or condition described herein.

### Cardiovascular Disease

In some embodiments, the compositions and methods described herein relate to the treatment or prevention of heart diseases, vascular diseases and/or cardiovascular diseases or disease of the cardiovascular system, e.g., an aneurysm (e.g., abdominal aortic aneurysm (AAA), thoracic aortic aneurysm (TAA) or cerebral aneurysm), hypertension, coronary artery disease, stroke, peripheral artery disease, cerebral vascular disease, diabetes-derived cardiovascular diseases/complications, congestive heart failure, acute and chronic heart failure, arterial hypertension, primary and secondary hypertension, coronary heart disease, stable and instable angina pectoris, myocardial ischemia, myocardial ischemia reperfusion injury, myocardial infarction, coronary microvascular dysfunction, microvascular obstruction, no-reflow-phenomenon, shock, atherosclerosis, coronary artery disease, peripheral artery disease, peripheral arterial disease, intermittent claudication, severe intermittent claudication, limb ischemia, critical limb ischemia, hypertrophy of the heart, cardiomyopathies of any etiology (such as, e.g., dilatative cardiomyopathy, restrictive cardiomyopathy, hypertrophic cardiomyopathy, ischemic cardiomyopathy), fibrosis of the heart, atrial and ventricular arrhythmias, transitory and/or ischemic attacks, apoplexy, ischemic and/or hemorrhagic stroke, preeclampsia, inflammatory cardiovascular diseases, metabolic diseases, obesity, diabetes, type-I-diabetes, type-II-diabetes, diabetes mellitus, peripheral and autonomic neuropathies, diabetic neuropathies, diabetic microangiopathies, diabetic retinopathy, diabetic ulcera at the extremities, gangrene, CREST-syndrome, hypercholesterolemia, hypertriglyceridemia, lipometabolic disorder, metabolic syndrome, increased levels of fibrinogen and low-density lipoproteins (i.e. LDL), increased concentrations of plasminogen-activator inhibitor 1 (PAI-1), as well as peripheral vascular and cardiac vascular diseases, peripheral circulatory disorders, primary and secondary Raynaud syndrome, disturbances of the microcirculation, arterial pulmonary hypertension, primary and secondary pulmonary hypertension, spasms of coronary and peripheral arteries, thromboses, thromboembolic diseases, edema-formation, such as pulmonary edema, brain-edema, renal edema, myocardial edema, myocardial edema associated with heart failure, restenosis after i.e. thrombolytic therapies, percutaneous-transluminal angioplasties (PTA), transluminal coronary angioplasties (PTCA), heart transplantations, lung transplantations, kidney transplantations, bypass-surgeries as well as micro- and macrovascular injuries (e.g., vasculitis), reperfusion-damage, arterial and venous thromboses, microalbuminuria, cardiac insufficiency, endothelial dysfunction. In the light of the present disclosure, heart failure includes more specific or related kinds of diseases such as acute decompensated heart failure, right heart failure, left heart failure, global insufficiency, ischemic cardiomyopathy, dilatative cardiomyopathy, congenital heart defect(s), valve diseases, heart failure related to valve diseases, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined valvular defects, inflammation of the heart muscle (myocarditis), chronic myocarditis, acute myocarditis, viral myocarditis, bacterial myocarditis, diabetic heart failure, alcohol-toxic cardiomyopathy, cardiac storage diseases, heart failure with preserved ejection fraction (HFpEF), diastolic heart failure, heart failure with reduced ejection fraction (HFrEF), systolic heart failure. In the context of the present disclosure, the terms atrial arrhythmias and ventricular arrhythmias also include more specific and related disease-entitites, such as: Atrial fibrillation, paroxysmal atrial fibrillation, intermittent atrial fibrillation, persistent atrial fibrillation, permanent atrial fibrillation, atrial flutter, sinus arrhythmia, sinus tachycardia, passive heterotopy, active heterotopy, replacement systoles, extrasystoles, disturbances in the conduction of impulses, sick-sinus syndrome, hypersensitive carotis-sinus, tachycardias, AV-node re-entry tachycardias, atrioventricular re-entry tachycardia, WPW-syndrome (Wolff-Parkinson-White syndrome), Mahaim-tachycardia, hidden accessory pathways/tracts, permanent junctional re-entry tachycardia, focal atrial tachycardia, junctional ectopic tachycardia, atrial re-entry tachycardia, ventricular tachycardia, ventricular flutter, ventricular fibrillation, sudden cardiac death. In the context of the present disclosure, the term coronary heart disease also include more specific or related diseases entities, such as: Ischemic heart disease, stable angina pectoris, acute coronary syndrome, instable angina pectoris, NSTEMI (non-ST-segement-elevation myocardial infarction), STEMI (ST-segement-elevation myocardial infarction), ischemic damage of the heart, arrhythmias, and myocardial infarction.

### Endothelial Dysfunction

In some embodiments, the compositions and methods described herein relate to the treatment or prevention of endothelial dysfunction or a disease or condition related to endothelial dysfunction. Endothelial dysfunction refers to a condition characterized by a deficiency in nitric oxide (NO) bioavailability in endothelial cells lining the lumen of the blood vessels, leading to an array of dysfunctional events to result in pathogenesis of cardiovascular diseases and other human diseases. The dysfunctional events include, but are not limited to, vasoconstriction due to loss of NO-mediated vasodilation, increased platelet activation and neutrophil adhesion, increased inflammatory responses due to upregulated inflammatory protein expression, and endothelial cell apoptosis. Endothelial dysfunction has been implicated in a large variety of human diseases such as, but not limited to, hypertension, aneurysms, diabetic vascular diseases/complications, obesity/metabolic syndrome, pulmonary hypertension, ARDS, and ischemia reperfusion injury of the heart/myocardial infarction.

### Inflammatory Diseases and Conditions

In some embodiments, the compositions and methods provided herein are useful for the treatment or prevention of inflammation. In certain embodiments, the compositions and methods described herein can be used for preventing or treating inflammation of any tissue and organs of the body, including musculoskeletal inflammation, cardiac inflammation, vascular inflammation, neural inflammation, digestive system inflammation, ocular inflammation, and inflammation of the reproductive system.

The compositions and methods described herein can be useful for treatment or prevention of a disease or condition associated with a pathological immune response, such as adult respiratory distress syndrome (ARDS) or systemic inflammatory response syndrome (SIRS).

Acute respiratory distress syndrome (ARDS) is a serious lung condition that causes low blood oxygen and lethal respiratory failure. Individuals who develop ARDS are usually ill due to another disease or a major injury. ARDS often develop following trauma, inhalation of harmful substances, or sepsis induced by bacterial and/or viral infections such as by SARS or SARS-CoV-2. In ARDS, fluid builds up inside the tiny air sacs of the lungs due to a primary pathological hallmark of endothelial dysfunction to cause blood vessels to leak, and surfactant breaks down. Surfactant is a foamy substance that keeps the lungs fully expanded so that a person can breathe. These changes prevent the lungs from filling properly with air and moving enough oxygen into the bloodstream and throughout the body. The lung tissue may scar and become stiff.

SIRS is a serious condition related to systemic inflammation, organ dysfunction, and organ failure. It is a subset of cytokine storm, in which there is abnormal regulation of various cytokines. SIRS is also closely related to sepsis and subjects that satisfy criteria for SIRS may also have a suspected or proven infection. SIRS may be generally manifested as a combination of vital sign abnormalities including fever or hypothermia, tachycardia, tachypnea, and leukocytosis or leukopenia. SIRS is nonspecific and can be caused by ischemia, inflammation, trauma, burns, infection, pancreatitis, stress, organ injury, major surgery, fractures, or several insults combined.

### Autoimmune Disease

The compositions and methods described herein can be used, for example, for preventing or treating an autoimmune disease, such as chronic inflammatory bowel disease, systemic lupus erythematosus, psoriasis, muckle-wells syndrome, rheumatoid arthritis, multiple sclerosis, or Hashimoto's disease; an allergic disease, such as a food allergy, pollenosis, or asthma. The compositions and methods described herein can be used, for example, as a pharmaceutical composition for preventing or treating an inflammatory disease such as an inflammatory disease of the gastrointestinal tract, such as pouchitis, a cardiovascular inflammatory condition, such as atherosclerosis, or an inflammatory lung disease, such as chronic obstructive pulmonary disease, fibrotic disease, or cystic fibrosis.

The compositions and methods described herein may be used to treat or prevent autoimmune conditions having an inflammatory component. Such conditions include, but are not limited to, acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, giant cell arteritis, goodpasture's syndrome, Grave's disease, Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, Ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, Lyme disease, morphea, psoriasis, sarcoidosis, scleroderma, ulcerative colitis, and vitiligo.

### Cancer

In some embodiments, the compositions and methods described herein relate to the treatment or prevention of cancer, since many types of the cancers are related to endothelial dysfunction, inflammation and/or oxidative stress. In some embodiments, any cancer can be treated using the methods described herein. Examples of cancers that may treated by compositions and methods described herein include, but are not limited to, cancer cells of the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be one of the following histological types, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; or hairy cell leukemia.

In some embodiments, the cancer comprises breast cancer (e.g., triple negative breast cancer).In some embodiments, the cancer comprises colorectal cancer (e.g., microsatellite stable (MSS) colorectal cancer). In some embodiments, the cancer comprises renal cell carcinoma. In some embodiments, the cancer comprises lung cancer (e.g., non-small cell lung cancer). In some embodiments, the cancer comprises bladder cancer. In some embodiments, the cancer comprises gastroesophageal cancer.

In some embodiments, the compositions and methods provided herein relate to the treatment of a leukemia. The term “leukemia” includes broadly progressive, malignant diseases of the hematopoietic organs/systems and is generally characterized by a distorted proliferation and development of leukocytes and their precursors in the blood and bone marrow. Non-limiting examples of leukemia diseases include acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemia.

In some embodiments, the compositions and methods provided herein relate to the treatment of a carcinoma. The term “carcinoma” refers to a malignant growth made up of epithelial cells tending to infiltrate the surrounding tissues, and/or resist physiological and non-physiological cell death signals and gives rise to metastases. Non-limiting exemplary types of carcinomas include, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti.

In some embodiments, the compositions and methods provided herein relate to the treatment of a sarcoma. The term “sarcoma” generally refers to a tumor which is made up of a substance like the embryonic connective tissue and is generally composed of closely packed cells embedded in a fibrillar, heterogeneous, or homogeneous substance. Sarcomas include, but are not limited to, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Additional exemplary neoplasias that can be treated using the compositions and methods described herein include Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, plasmacytoma, colorectal cancer, rectal cancer, and adrenal cortical cancer.

In some embodiments, the cancer treated is a melanoma. The term “melanoma” is taken to mean a tumor arising from the melanocytic system of the skin and other organs. Non-limiting examples of melanomas are Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

Particular categories of tumors that can be treated using compositions and methods described herein include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, pulmonary squamous cell carcinoma, basal cell carcinoma, adenocarcinoma (well differentiated, moderately differentiated, poorly differentiated or undifferentiated), bronchioloalveolar carcinoma, renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma, plasmacytoma, colorectal cancer, and rectal cancer.

Cancers treated in certain embodiments also include precancerous lesions, e.g., actinic keratosis (solar keratosis), moles (dysplastic nevi), acitinic chelitis (farmer's lip), cutaneous horns, Barrett's esophagus, atrophic gastritis, dyskeratosis congenita, sideropenic dysphagia, lichen planus, oral submucous fibrosis, actinic (solar) elastosis and cervical dysplasia.

Tumors treated in some embodiments include non-cancerous or benign tumors, e.g., of endodermal, ectodermal or mesenchymal origin, including, but not limited to cholangioma, colonic polyp, adenoma, papilloma, cystadenoma, liver cell adenoma, hydatidiform mole, renal tubular adenoma, squamous cell papilloma, gastric polyp, hemangioma, osteoma, chondroma, lipoma, fibroma, lymphangioma, leiomyoma, rhabdomyoma, astrocytoma, nevus, meningioma, and ganglioneuroma.

### Exemplary Embodiments

Embodiment 1. A miRNA inhibitor comprising a nucleic acid that is at least 80% identical to any one of SEQ ID NOs: 1-19.

Embodiment 2. The miRNA inhibitor of embodiment 1, wherein the miRNA inhibitor comprises a nucleic acid that is at least 50% (e.g., 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 90%) identical to any one of SEQ ID NOs: 1-19.

Embodiment 3. The miRNA inhibitor of embodiment 1, wherein the miRNA inhibitor comprises a nucleic acid that is at least 95% identical to any one of SEQ ID NOs: 1-19.

Embodiment 4. The miRNA inhibitor of embodiment 1, wherein the miRNA inhibitor comprises a nucleic acid that is at least 98% identical to any one of SEQ ID NOs: 1-19.

Embodiment 5. The miRNA inhibitor of embodiment 1, wherein the miRNA inhibitor comprises a nucleic acid that is any one of SEQ ID NOs: 1-19.

Embodiment 6. The miRNA inhibitor of any one of embodiments 1-5, wherein the nucleic acid comprises a chemical modification.

Embodiment 7. The miRNA inhibitor of embodiment 6, wherein the chemical modification is a 2′-O-methylated nucleoside (2′OMe), a 2′-fluoro oligonucleotide (2′F), a 2′-O-methoxyethyl oligonucleotide (2′MOE), a phosphorodiamidate morpholino oligonucleotide (PMO), a peptide nucleic acid (PNA), a phosphorothioate bond (PS), a locked nucleic acid (LNA), a hydrophobic moiety, a naphthyl modifier, a non-nucleotide N,N-diethyl-4-(4-nitronaphthalen-1-ylazo)-phenylamine (ZEN), or a cholesterol moiety.

Embodiment 8. The miRNA inhibitor of embodiment 7, wherein the chemical modification is a 2′-O-methylated nucleoside (2′OMe).

Embodiment 9. The miRNA inhibitor of embodiment 7, wherein the chemical modification is a 2′-fluoro oligonucleotide (2′F).

Embodiment 10. The miRNA inhibitor of embodiment 7, wherein the chemical modification is a 2′-O-methoxyethyl oligonucleotide (2′MOE).

Embodiment 11. The miRNA inhibitor of embodiment 7, wherein the chemical modification is a phosphorodiamidate morpholino oligonucleotide (PMO).

Embodiment 12. The miRNA inhibitor of embodiment 7, wherein the chemical modification is a peptide nucleic acid (PNA).

Embodiment 13. The miRNA inhibitor of embodiment 7, wherein the chemical modification is a phosphorothioate bond (PS).

Embodiment 14. The miRNA inhibitor of embodiment 7, wherein the chemical modification is a locked nucleic acid (LNA).

Embodiment 15. The miRNA inhibitor of embodiment 7, wherein the chemical modification is a hydrophobic moiety.

Embodiment 16. The miRNA inhibitor of embodiment 7, wherein the chemical modification is a naphthyl modifier.

Embodiment 17. The miRNA inhibitor of embodiment 7, wherein the chemical modification is a cholesterol moiety.

Embodiment 18. The miRNA inhibitor of any one of embodiments 1-17, wherein the nucleic acid is complementary to any one of SEQ ID NOs: 20-38.

Embodiment 19. The miRNA inhibitor of any one of embodiments 1-18, wherein the miRNA inhibitor binds to a miRNA comprising a nucleic acid that is at least 50% (e.g., 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 90%) identical to any one SEQ ID NOs: 20-38.

Embodiment 20. The miRNA inhibitor of any one of embodiments 1-18, wherein the miRNA inhibitor binds to a miRNA comprising a nucleic acid that is at least 90% identical to any one SEQ ID NOs: 20-38.

Embodiment 21. The miRNA inhibitor of any one of embodiments 1-18, wherein the miRNA inhibitor binds to a miRNA comprising a nucleic acid that is at least 95% identical to any one SEQ ID NOs: 20-38.

Embodiment 22. The miRNA inhibitor of any one of embodiments 1-18, wherein the miRNA inhibitor binds to a miRNA comprising a nucleic acid that is at least 98% identical to any one SEQ ID NOs: 20-38.

Embodiment 23. The miRNA inhibitor of any one of embodiments 1-18, wherein the miRNA inhibitor binds to a miRNA comprising a nucleic acid that is any one SEQ ID NOs: 20-38.

Embodiment 24. The miRNA inhibitor of any one of embodiments 1-23, wherein the miRNA inhibitor is at least 10 nucleotides in length.

Embodiment 25. The miRNA inhibitor of any one of embodiments 1-23, wherein the miRNA inhibitor is at least 18 nucleotides in length.

Embodiment 26. The miRNA inhibitor of any one of embodiments 1-25, wherein the miRNA inhibitor is no more than 30 nucleotides in length.

Embodiment 27. The miRNA inhibitor of any one of embodiments 1-25, wherein the miRNA inhibitor is no more than 22 nucleotides in length.

Embodiment 28. A miRNA inhibitor that competes with a miRNA inhibitor of any one of embodiments 1-27 for binding to a miRNA comprising a nucleic acid that is any one of SEQ ID NOs: 20-38.

Embodiment 29. A vector comprising the miRNA inhibitor of any one of embodiments 1-28.

Embodiment 30. A pharmaceutical composition comprising miRNA inhibitor of any one of embodiments 1-28 or a vector of embodiment 29.

Embodiment 31. The pharmaceutical composition of embodiment 30, further comprising a pharmaceutically acceptable carrier.

Embodiment 32. The pharmaceutical composition of embodiment 30 or 31, for use in preventing, inhibiting, treating, or reducing aneurysms.

Embodiment 33. The pharmaceutical composition of embodiment 32, wherein the aneurysm is abdominal aortic aneurysm, cerebral aneurysm, or thoracic aortic aneurysm.

Embodiment 34. A method of preventing, inhibiting, treating, or reducing aneurysms in a subject, comprising administering to the subject a pharmaceutical composition comprising an miRNA inhibitor comprising a nucleic acid sequence that binds to at least a portion of a miR-192-5p sequence.

Embodiment 35. The method of embodiment 34, wherein the pharmaceutical composition comprises a vector.

Embodiment 36. The method of embodiment 34 or 35, wherein miRNA inhibitor suppresses the function of the mature miR-192-5p.

Embodiment 37. A method of preventing, inhibiting, treating, or reducing aneurysms in a subject, comprising administering to the subject a pharmaceutical composition comprising an miRNA inhibitor comprising a nucleic acid sequence set forth in Tables 1-4.

Embodiment 38. The method of embodiment 37, wherein the pharmaceutical composition is administered subcutaneously.

Embodiment 39. The method of embodiment 37, wherein the pharmaceutical composition is administered parenterally.

Embodiment 40. The method of any one of embodiments 37-39, wherein the aneurysm is abdominal aortic aneurysm, cerebral aneurysm, or thoracic aortic aneurysm.

Embodiment 41. The method of any one of embodiments 37-40, further comprising conjointly administering to the subject an additional therapeutic agent.

Embodiment 42. The method of embodiment 41, wherein the additional therapeutic agent is a folate compound and/or a calcium channel blocker.

Embodiment 43. A method of reversing vascular remodeling, comprising administering to the subject a pharmaceutical composition comprising an miRNA inhibitor comprising a nucleic acid sequence that binds to at least a portion of a miR-192-5p sequence, wherein vascular remodeling is characterized by inflammation, matrix degradation, adventitial hypertrophy, medial elastin degradation and flattening, and/or formation of intra-lumen thrombi.

Embodiment 44. A method of decreasing reactive oxygen species production, comprising administering to the subject a pharmaceutical composition comprising an miRNA inhibitor comprising a nucleic acid sequence that binds to at least a portion of a miR-192-5p sequence.

Embodiment 45. A method of decreasing reactive oxygen species production, comprising administering to the subject a pharmaceutical composition comprising an miRNA inhibitor comprising a nucleic acid sequence set forth in Tables 1-4.

Embodiment 46. A method of restoring endothelial nitric oxide synthase (eNOS) coupling activity, comprising administering to the subject a pharmaceutical composition comprising a miRNA inhibitor comprising a nucleic acid sequence that binds to at least a portion of a miR-192-5p sequence.

Embodiment 47. A method of restoring endothelial nitric oxide synthase (eNOS) coupling activity, comprising administering to the subject a pharmaceutical composition comprising an miRNA inhibitor comprising a nucleic acid sequence set forth in Tables 1-4.

Embodiment 48. A method of preserving nitric oxide (NO) bioavailability, comprising administering to the subject a pharmaceutical composition comprising an miRNA inhibitor comprising a nucleic acid sequence that binds to at least a portion of a miR-192-5p sequence.

Embodiment 49. A method of preserving nitric oxide (NO) bioavailability, comprising administering to the subject a pharmaceutical composition comprising an miRNA inhibitor comprising a nucleic acid sequence set forth in Tables 1-4.

Embodiment 50. A method of treating or preventing aneurysm (abdominal aortic aneurysm (AAA), thoracic aortic aneurysm (TAA) or cerebral aneurysm), hypertension, acute respiratory distress syndrome (ARDS), or any other diseases associated with endothelial dysfunction in a subject, comprising administering to the subject an miRNA inhibitor comprising a nucleic acid that is at least 50% (e.g., 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 90%) identical to any one of SEQ ID NOs: 1-19.

Embodiment 51. A method of treating or preventing aneurysm (abdominal aortic aneurysm (AAA), thoracic aortic aneurysm (TAA) or cerebral aneurysm), hypertension, acute respiratory distress syndrome (ARDS), or any other diseases associated with endothelial dysfunction in a subject, comprising administering to the subject a miRNA inhibitor comprising a nucleic acid sequence that binds to at least a portion of a miR-192-5p sequence.

Embodiment 52. The method of embodiment 51, wherein the miRNA inhibitor has at least 50% (e.g., 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 90%) complementarity to a portion of miR-192-5p.

Embodiment 53. The method of embodiment 51, wherein the miRNA inhibitor has at least 95% complementarity to a portion of miR-192-5p.

Embodiment 54. The method of embodiment 51, wherein the miRNA inhibitor has at least 99% complementarity to a portion of miR-192-5p.

Embodiment 55. The method of embodiment 51, wherein the miRNA inhibitor has 100% complementarity to a portion of miR-192-5p.

Embodiment 56. The method of any one of embodiments 51-55, wherein the miRNA inhibitor suppresses the function of the mature miR-192-5p.

Embodiment 57. The miRNA inhibitor of embodiment 7, wherein the chemical modification is a non-nucleotide N,N-diethyl-4-(4-nitronaphthalen-1-ylazo)-phenylamine (ZEN).

### EXAMPLES

The invention now being generally described, it will be more readily understood by reference to the following examples which are included merely for purposes of illustration of certain aspects and embodiments of the present invention, and are not intended to limit the invention.

Applicant has shown herein that endothelial specific dihydrofolate reductase (DHFR) deficiency underlies eNOS uncoupling and formation of abdominal aortic aneurysm (AAA). Here, a novel role of miR-192-5p in mediating NOX-dependent DHFR deficiency and AAA formation is investigated. miR-192-5p is predicted to target DHFR. Intriguingly, hsa-miR-192-5p mRNA expression was substantially upregulated in human AAA patients. In human aortic endothelial cells (HAECs) exposed to hydrogen peroxide (H₂O₂), hsa-miR-192-5p expression was significantly upregulated. This was accompanied by a marked downregulation in DHFR mRNA and protein expression, which was restored by hsa-miR-192-5p specific inhibitor. miR-192-5p expression was markedly upregulated in Ang II infused hph-1 mice, which was attenuated in hph-1-NOX1, hph-1-NOX2, hph-1-p47phox, and hph-1-NOX4 double mutant mice where AAA incidence was also abrogated, indicating a downstream effector role of miR-192-5p following NOX activation. In vivo treatment with mmu-miR-192-5p as inhibitor attenuated expansion of abdominal aortas in Ang II infused hph-1 mice as defined by echocardiography and post-mortem inspection. It also reversed features of vascular remodeling including matrix degradation, adventitial hypertrophy and formation of intra-lumen thrombi. These animals had restored DHFR mRNA and protein expression, attenuated superoxide production, recoupled eNOS, and preserved NO bioavailability. In conclusion, our data herein demonstrate a critical role of miR-192-5p in mediating NOX-dependent DHFR deficiency and AAA formation, inhibition of which is robustly effective in attenuating development of AAA. Since the mouse and human miR-192-5p sequences are identical, the miR-192-5p inhibitors are readily translatable into novel therapeutics for the treatment of AAA.

### Example 1. Materials and Methods

The following materials and methods were used in the Examples 2-7.

### Reagents:

Unless otherwise noted, all chemicals were purchased from Millipore-Sigma (Burlington, MA, USA) in the highest purity. Isoflurane was obtained from Piramal Healthcare (Bethlehem, PA, USA).

### Human AAA Samples:

The aortic aneurysmal tissue samples of human AAA were obtained from the NIH NDRI (National Disease Research Interchange) program with approved IRB (Institutional Review Board) protocol, and the control subjects were those of donors died of sudden causes, but without aneurysms (age: 73.1±11.4 years old for control vs. 72.5±10.8 years old for AAA; 11 males and 4 females for control vs. 10 males and 5 females for AAA).

Cell Culture and miRNA Inhibitors Transfection

Human aortic endothelial cells (HAECs) of passages 3 to 7 donated from 2 males (aged 49 and 50; Lonza; Walkersville, MD) were cultured in EGM2 media supplemented with 10% (v/v) fetal bovine serum (FBS) and supplements (Hydrocortisone, hFGF-B, VEGF, R3-IGF-1, Ascorbic Acid, hEGF, GA-1000 and Heparin, all reagents from Lonza, USA). Cells were grown in a humidified atmosphere at 5% C02 and 37° C. miRNA inhibitors and negative controls (50 μmol/L; 100 μmol/well in six-well plate, Life Technologies Corporation, Grand Island, NY 14072, USA) transfection into HAECs was performed using Lipofectamine RNAiMAX (Thermo Fisher Scientific) according to the manufacturers' instructions. Transfection was performed 48 h prior to being stimulated with 100 μmol/L H₂O₂ for 24 h in HAECs. Then cells were harvested for subsequent analyses of has-miR-192 expression, and DHFR mRNA and protein expression.

RNA Extraction, microRNA-Specific cDNA Synthesis, and qRT-PCR of microRNA

Total RNAs were extracted from HAECs using TRIzol® (Invitrogen Corp., Carlsbad, CA, USA) according to the manufacturer's instructions. The first-strand cDNA was synthesized from RNA samples using the Mir-X miRNA First-Strand Synthesis kit (Clontech Laboratories, Inc., A Takara Bio Company, Mountain View, CA, USA) according to the manufacturer's instructions. Primers were designed on the basis of miRBase sequence (hsa-miR-192-5p, MIMAT0000222, miRBase). Variability in the initial quantities of cDNA was normalized relative to the abundance of U6 after amplification (supplied by Clontech Laboratories, Inc.), and the data were expressed as fold changes. qRT-PCR of microRNAs were conducted with the SYBR qRT-PCR kit (Clontech Laboratories, Inc., A Takara Bio Company, Mountain View, CA, USA) according to the manufacturer's instructions.

Real-Time RT-PCR Determination of mRNA Expression

Real-time RT-PCR amplification of DHFR mRNA was performed as previously described. Each PCR reaction was done in triplicates and quantification was performed with the efficiency-corrected 2^{ΔΔCt} method using the housekeeping gene glyceraldehyde 3-phosphate dehydrogenase (GAPDH) as endogenous control. The primers used for human DFHR are: Sense: 5′-CTTTCCAGAAGTCTAGATGC-3′; Antisense: 5′-GGTATCTGATAGAGACAAGAG-3′. The primers used for GAPDH are: Sense: 5′-AACGGGAAGCTTGTCATCAATGGAAA-3′; Antisense: 5′-GCATCAGCAGAGGGGGCAGAG-3′. Data are presented as fold changes of control group. All primers used for real time-PCR were synthesized by Integrated DNA Technologies (San Diego, CA).

### Western Blotting

Twenty micrograms of proteins were subjected to SDS-PAGE (10% gel) and transferred to nitrocellulose membrane (Amersham Inc, Marlborough, MA, USA). After 1 h blocking in PBS containing 0.1% Tween-20 and 5% (w/v) non-fat dry milk, the membranes were then incubated with primary antibodies for DHFR (1:300, Novus Biologicals, Littleton, CO, USA), and actin (1:10,000, Sigma-Millipore, address) respectively.

Osmotic Pump Infusion of Ang II into Hph-1 Mice and Hph-1-NOX Isoform/Subunit Double Mutant Mice

All animals and experimental procedures were approved by the Institutional Animal Care and Usage Committee at the University of California Los Angeles. Homozygote hph-1, and double mutants of hph-1-NOX1, hph-1-NOX2, hph-1-p47phox, and hph-1-NOX4 mice generated in house, were maintained as described before. Animals were anesthetized with isoflurane in an isoflurane chamber, and then moved to a nose cone supplying 1.5-2% isoflurane to maintain the anesthetic state. A small area between the shoulder blades in the back of the mice was removed of hair, and then disinfected with iodine solution. A small incision was made at the site, followed by the insertion of an osmotic pump (Alzet, model 2002, Cupertino, CA, USA) containing Ang II (0.7 mg/kg/day) in a delivery solution (3.8 mL H₂O, 120 μl 5 M NaCl, 40 μl acetic acid) under the skin to the left flank. Surgical staples were used to close the wound. The animals were placed in a heated chamber for recovery. The locked nucleic acid (LNA)-mmu-miR-192-5p inhibitors were synthesized by Exiqon (now a QIAGEN company, Germantown, MD, USA), and used to inject (30 mg/kg) into hph-1 mice subcutaneously on the first day and the third day after implantation of Ang II pumps. The LNA-negative-control was injected into the animals as a control group.

### Tissue Collection

After 2 weeks of Ang II infusion, animals were euthanized with C02. The aortas were rapidly removed from the body, rinsed with ice cold modified Krebs/HEPES buffer (KHB: 99 mmol/L NaCl; 4.7 mmol/L KCl; 1.2 mmol/L MgSO₄; 1.0 mmol/L KH₂PO₄; 2.5 mmol/L CaCl₂); 25 mmol/L NaHCO₃; 5.6 mmol/L D-glucose; 20 mmol/L NaHEPES), and cleaned of connective tissue and fat on ice. Determination of AAA incidence was made by ultrasound analysis of aortic size and post-mortem inspection. External diameters of isolated aortas were also measured with ruler. A small section (about 2 mm) of the suprarenal aorta was collected for subsequent histological analyses.

Isolation of Endothelial Cells from Aorta

Endothelial cells (ECs) were isolated from aortas as previously described. Briefly, freshly isolated aortas were cut into small sections (about 2 mm) and digested in PBS containing collagenase (0.6 mg/mL) for 20 min at 37° C. The aortic rings were then gently shaken in the digestion buffer to remove ECs. The ECs were collected via centrifugation at 1,000 g for 3 min at 4° C. and lysed with lysis buffer for analysis of DHFR protein expression by Western blotting, or lysed with TRIzol® for analysis of DHFR mRNA and miR-192-5p expression by RT-PCR.

In Vivo Treatment of Hph-1 Mice with Mmu-miR-192-5p Inhibitors

The locked nucleic acid (LNA)-mmu-miR-192-5p inhibitors were synthesized by Exiqon (now a QIAGEN company, Germantown, MD, USA), and used to inject (30 mg/kg each time) into hph-1 mice subcutaneously on the first day and the third day after implantation of Ang II pumps. The LNA-negative-control was injected into the animals as a control group. mmu-miR-192-5p mirVana® miRNA inhibitor and negative control for in vivo experimentation were synthesized by ThermoFisher (Grand Island, NY, USA). mmu-miR-192-5p mirVana® miRNA inhibitor or negative control was injected intravenously into hph-1 mice via tail vein on the first day and the third day (2.5 mg/kg each time) after implantation of Ang II pumps.

### Histological Analyses

H&E staining was performed by the Translational Pathology Core Laboratory (TPCL) Core Facility at UCLA using standard protocols, following sectioning. For VVG staining to visualize elastin fibers, paraffin embedded tissue sections were de-paraffinized by sequential washes in xylene (2×), descending alcohol from 100% to 50%, then into distilled water. Sections were then stained in Verhoeff's solution for 70 min, followed by differentiation in 2% ferric chloride for 90 s. Sections were then incubated with 5% sodium thiosulfate for 60 s, followed by counterstaining with Van Gieson's solution and dehydration with 95% and 100% alcohol, and finally washed in xylene. After drying, the tissues were mounted with Permount (Fisher Scientific, Pittsburgh, PA, USA) and images captured using Nikon TE2000-U fluorescent microscope.

### DHE Detection of Aortic ROS Production.

As described previously, dihydroethidium (DHE) staining was used to examine the efficacy of miR-192-5p inhibitors on overall vascular ROS production in situ. DHE is a cell permeable dye that is oxidized by superoxide to ethidium bromide, which subsequently interacts with DNA and is trapped within the nuclei of the cells. Aortas were freshly harvested and rings of aortas were embedded in OCT compound, immediately frozen at −20° C. and sectioned. Seven micrometer thick-frozen sections were rinsed briefly in modified Krebs/HEPES buffer (KHB, contents described as above) to remove OCT compound, and then covered in DHE (2 μmol/L) solution for incubation in a lightproof humidified container at 37° C. for 30 min. Slides were then washed with KHB 3 times, mounted by ProLong Gold Antifade reagent (Invitrogen Corp., Carlsbad, CA, USA) and imaged with a Nikon TE2000-U fluorescent microscope at excitation and emission wavelengths of 488 nm and 610 nm respectively.

### Electron Spin Resonance (ESR) Determination of Superoxide Levels

As previously described, freshly isolated aortas were homogenized on ice in lysis buffer supplemented with protease inhibitor cocktail (1:100), and centrifuged at 12,000 g for 15 min. Protein content of the supernatant was determined using a protein assay kit (Bio-Rad, Irvine, CA, USA). Five g of proteins were mixed with ice-cold and nitrogen bubbled KHB containing diethyldithiocarbamic acid (5 moL/L), deferoxamine (25 moL/L), and the freshly prepared superoxide specific spin trap methoxycarbonyl-2,2,5,5-tetramethylpyrrolidine (CMH, 500 moL/L, Axxora, San Diego, CA, USA). The mixture was then loaded into a glass capillary (Kimble, Dover, OH, USA), and assayed using the electron spin resonance (ESR) spectrometer (eScan, Bruker, Billerica, MA, USA) for superoxide production. A second measurement was taken with the addition of PEG-SOD (100 U/mL). To assess eNOS uncoupling activity, a third measurement was made with the addition of L-NAME (100 moL/L). The ESR settings used were: center field, 3480; sweep width, 9 G; microwave frequency, 9.78 GHz; microwave power, 21.02 mW; modulation amplitude, 2.47 G; 512 points of resolution; receiver gain, 1000.

### Electron Spin Resonance (ESR) Determination of Nitric Oxide (NO) Bioavailability

Aortic NO bioavailability was determined by ESR. Freshly isolated aortas were cut into 2 mm rings, and then incubated in freshly prepared NO specific spin trap Fe²⁺ (DETC)₂ (0.5 mmol/L) colloid in nitrogen bubbled, modified Krebs/HEPES buffer at 37° C. for 60 min, in the presence of calcium ionophore A23187 (10 μmol/L). The aortic rings were snap frozen in liquid nitrogen and loaded into a finger Dewar for measurement with ESR spectrophotometer (eScan, Bruker, Billerica, MA, USA). The instrument settings used were as the followings: Center field, 3440; Sweep width, 100 G; microwave frequency, 9.796 GHz; microwave power 13.26 mW; modulation amplitude, 9.82 G; 512 points of resolution; and receiver gain 356.

### Ultrasound Imaging of Abdominal Aortas

Ultrasound determination of abdominal aortic size was performed. Animals were anesthetized with isoflurane and placed on a temperature-controlled table. Hair was removed from the abdomen using a hair removal cream, and preheated ultrasound transmission gel was applied onto the abdomen area. An ultrasound probe (Velvo 2100, echocardiograph, MS-400) was placed on the gel to visualize aorta transversely. The aorta was identified using Doppler measurement for the presence of pulsatile flow. Consistent localization for image acquisition was ensured by visualizing the aorta immediately superior to the branch of the left renal artery in all of the animals.

### Statistical Analysis

All statistical analyses were carried out with the Prism software. Comparison between two groups was performed using the student's t-test. Comparisons between multiple groups were done using one-way ANOVA, followed by the Newman-Keuls post-hoc test. Comparisons of the incidence rates of AAA among different animal groups were performed using chi-squared test. Statistical significance was set at p<0.05. All grouped data are presented as Mean±SEM.

### Example 2. Hydrogen Peroxide Downregulated DHFR Expression while Upregulating miR-192-5p Expression

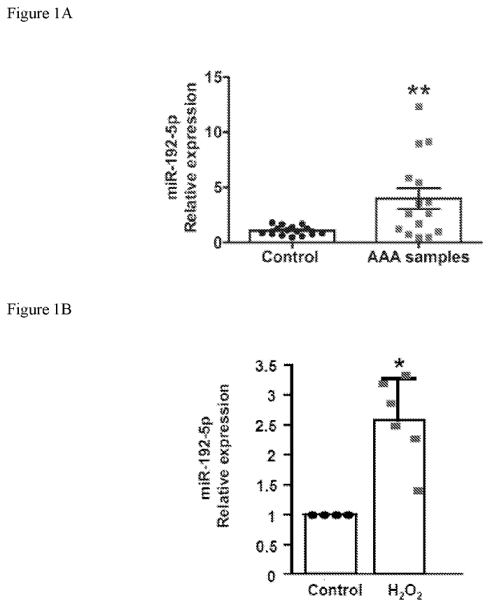

The present study examined an intermediate role of miR-192-5p in NOX-dependent modulation of DHFR to impact on AAA formation. First, the study examined expression of miR-192-5p in aortic aneurysmal tissues of human AAA. Intriguingly, the expression of hsa-miR-192-5p was substantially upregulated in the aortic aneurysmal tissues of human AAA patients comparing to donor controls (FIG. 1A). The aortic aneurysmal tissue samples of human AAA and the control non-AAA subjects were obtained from the NIH NDRI program with approved IRB protocol (age: 73.1±11.4 years old for control vs. 72.5±10.8 years old for AAA; 11 males and 4 females for control vs. 10 males and 5 females for AAA). In H₂O₂ (100 μM, 24 h) treated HAECs, hsa-miR-192-5p expression was also significantly upregulated (FIG. 1i). Interestingly, DHFR is a putative target of miR-192-5p by TargetScan (http://www.targetscan.org/). Of note, miR-192-5p was shown to decrease DHFR protein abundance in human colon cancer cell lines, and inhibit medulloblastoma cell proliferation by binding to DHFR 3-UTR. It was therefore hypothesize that miR-192-5p, with increased expression in both aortic aneurysmal tissues of human AAA patients and H₂O₂ treated HAECs, might mediate H₂O₂ downregulation of DHFR in vivo to induce AAA formation.

### Example 3. Silencing of miR-192-5p with Specific Inhibitors Restored DHFR Expression in Endothelial Cells

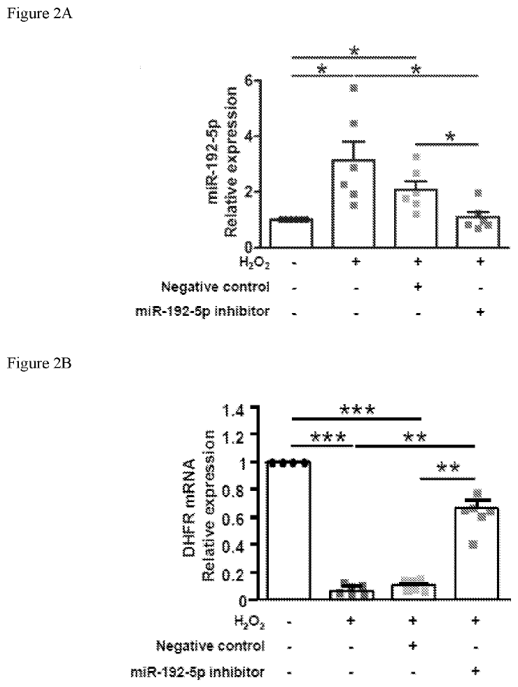

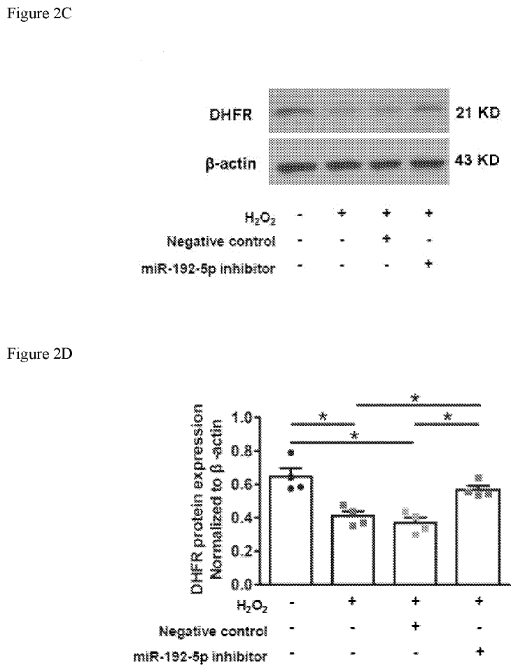

To examine whether miR-192-5p downregulates DHFR in ECs, HAECs were exposed to H₂O₂ after transfection of miR-192-5p inhibitors for 48 h. As shown in FIG. 2A, miR-192-5p expression levels were reduced in miR-192-5p-specific inhibitor treated HAECs. Furthermore, hsa-miR-192-5p-specific inhibitor markedly restored DHFR mRNA (FIG. 2B) and protein expression (FIGS. 2C and 2D), indicating an intermediate role of miR-192-5p in H₂O₂ induced DHFR deficiency.

### Example 4. miR-192-5p Expression in Ang H Infused Hph-1 Mice: NOX-Dependent Upregulation

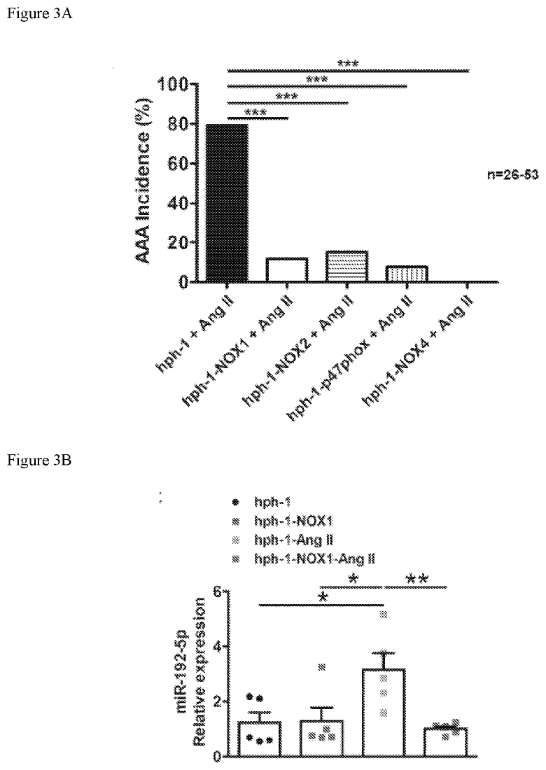

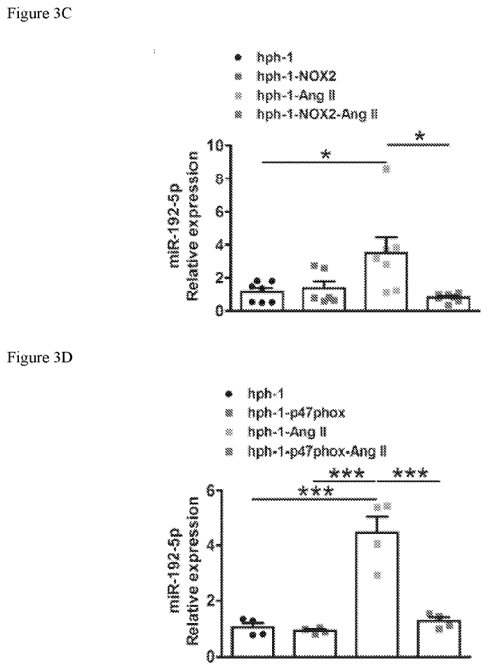

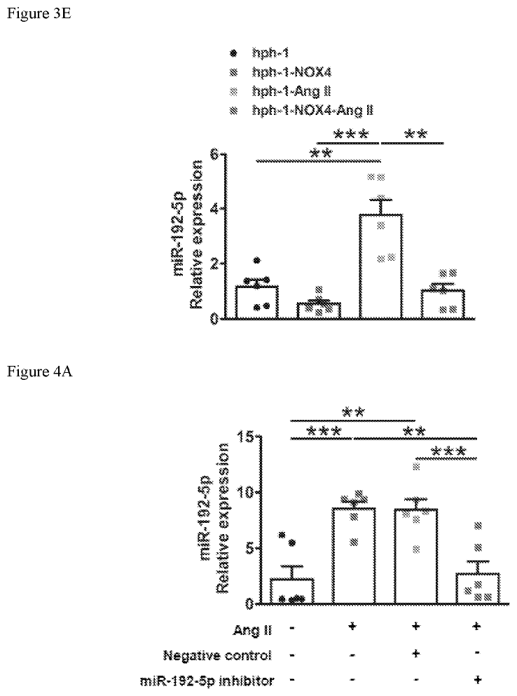

Of note, miR-192-5p is highly conserved among species. Therefore, the study explored a potential role of miR-192-5p in AAA formation in Ang II-infused hph-1 mice, via predicted downregulation of DHFR in vivo. It has been previously shown that DHFR deficiency lies downstream of NOX isoforms 1, 2 or 4 activation in Ang II-infused hph-1 mice, resulting in eNOS uncoupling to induce AAA formation. The incidence of AAA was substantially reduced, with significant difference by chi-square test, from 79.2% in AngII-infused hph-1 animals to 11.8%, 15.2%, 7.7% and 0% in hph-1-NOX1, hph-1-NOX2, hph-1-p47phox, and hph-1-NOX4 double mutant animals, respectively (FIG. 3A and Table 4) (combined data from the present study and the previous work). The miR-192-5p expression was significantly upregulated in Ang II infused hph-1 mice compared to WT mice, which was abrogated in hph-1-NOX1 (FIG. 3B), hph-1-NOX2 (FIG. 3C), hph-1-p47phox (FIG. 3D) and hph-1-NOX4 (FIG. 3E) double mutant mice, indicating a downstream role of miR-192-5p following NOX activation.

Table 4 shows miR-192-5p serves as a downstream effector of NOXs in mediating AAA formation. Ang II was infused into hph-1, hph-1-NOX1, hph-1-NOX2, hph-1-p47phox, and hph-1-NOX4 double mutant animals prior to phenotyping for AAA and isolation of aortic endothelial cells for detection of miR-192-5p expression levels. The data shows the actual numbers of animals in each experimental groups with and without AAA, combined with our original data reported in Siu K L et al. Redox Biol. 2017; 11:118-125. The incidence of AAA was greatly reduced from 79.3% in Ang II-infused hph-1 mice to 11.8%, 15.2%, 7.7% and 0% in hph-1-NOX1, hph-1-NOX2, hph-1-p47phox, and hph-1-NOX4 double mutant animals respectively.

TABLE 5

| | AAA | No AAA | Incidence |
|---|---|---|---|
| | (n) | (n) | (%) |
| hph-1 + Ang II | 42 | 11 | 79.2 |
| hph-1-NOX1 + Ang II | 4 | 30 | 11.8 |
| hph-1-NOX2 + Ang II | 7 | 39 | 15.2 |
| hph-1-p47phox + Ang II | 2 | 24 | 7.7 |
| hph-1-NOX4 + Ang II | 0 | 39 | 0.0 |

### Example 5. miR-192-5p Inhibitors Restored DHFR Expression in Ang H Infused Hph-1 Mice

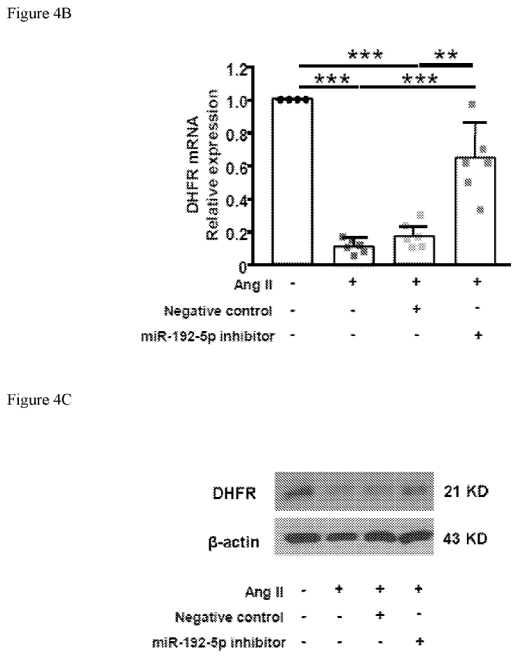

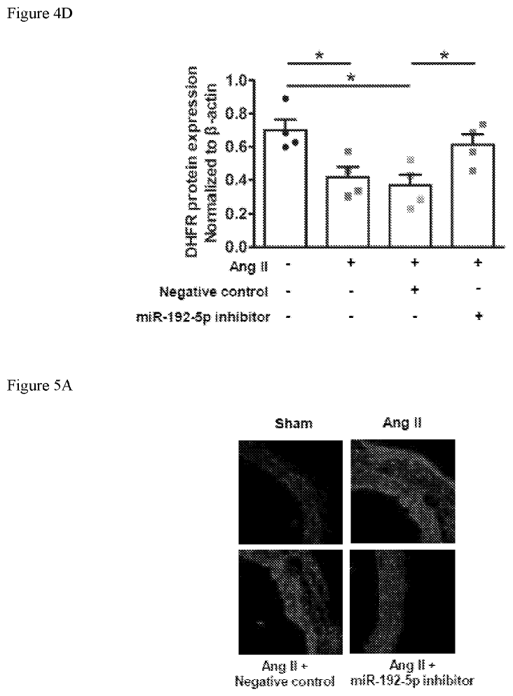

Of note, the mmu-miR-192-5p specific inhibitors were used to examine whether inhibition of miR-192-5p restores DHFR expression in Ang II infused hph-1 mice. As demonstrated in FIG. 4A, qRT-PCR analysis of miR expression indicated that mmu-miR-192-5p specific inhibitors decreased mmu-miR-192-5p expression in ECs isolated from aortas of Ang II infused hph-1 mice. Ang II infused hph-1 mice had significant decreased DHFR mRNA (FIG. 4B) and protein expression (FIGS. 4C and 4D) compared to hph-1 mice, while mmu-miR-192-5p inhibitors substantially restored DHFR mRNA (FIG. 4B) and protein expression (FIGS. 4C and 4D) comparing to Ang II infused hph-1 mice in ECs isolated from aortas.

### Example 6. miR-192-5p Inhibitors Diminished Endothelial Superoxide Production, Recoupled eNOS and Restored NO Bioavailability in Ang II Infused Hph-1 Mice

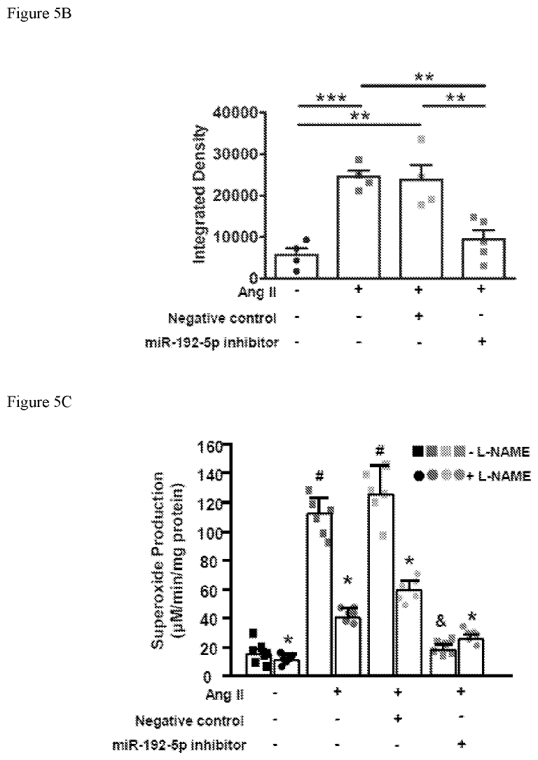

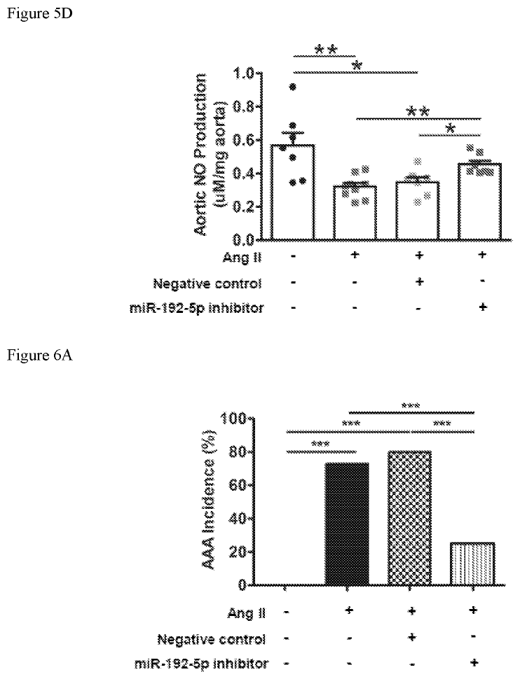

It was previously shown that endothelial DHFR deficiency leads to a reduction in H₄B bioavailability and consequent endothelial nitric oxide synthase (eNOS) uncoupling to result in development of AAA. Aortic production of total ROS detected by DHE staining was markedly increased in Ang II infused hph-1 mice comparing to untreated hph-1 mice, which was significantly attenuated in Ang II infused hph-1 mice with mmu-miR-192-5p inhibitors (FIGS. 5A and 5B). In addition, aortas were harvested and subjected to electron spin resonance (ESR) determination of superoxide production in the presence or absence of L-NAME, an inhibitor of NOS. If eNOS is functional and coupled, its inhibition by L-NAME to remove the buffering effect of NO will increase the measured superoxide. However, if eNOS is dysfunctional and uncoupled, it produces superoxide. Therefore, inhibition with L-NAME will reduce measured superoxide. There is an increased superoxide production in Ang II infused hph-1 mice, and this increase was inhibited by L-NAME, indicating that uncoupled eNOS is the enzymatic source of the superoxide production (FIG. 5C), as it was previously shown. L-NAME-sensitive superoxide production, reflective of eNOS uncoupling activity, was completely attenuated by miR-192-5p inhibitors (FIG. 5C). Notably, NO bioavailability was decreased in Ang II infused hph-1 mice, which was also substantially restored by miR-192-5p inhibitors (FIG. 5D). These results show that miR-192-5p inhibitors could improve the coupling state of eNOS, reduce ROS production and restore NO bioavailability in Ang II infused hph-1 mice via restoration of DHFR expression.

### Example 7. miR-192-5p Inhibitors Attenuated AAA Formation in Ang II Infused Hph-1 Mice

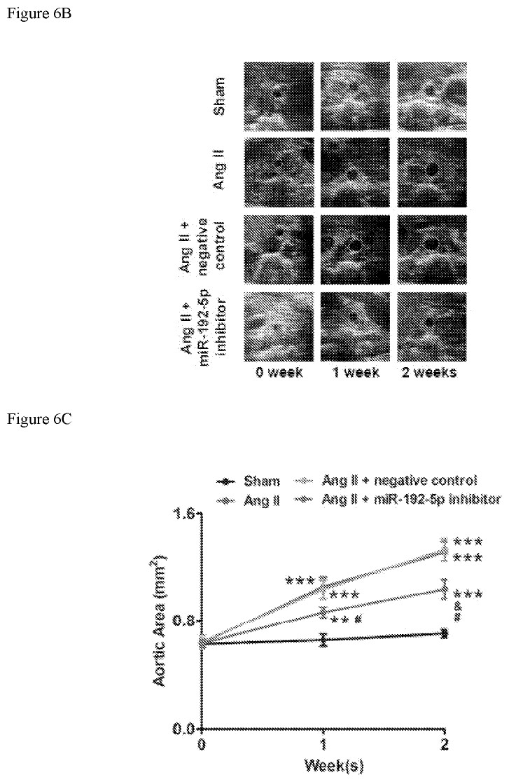

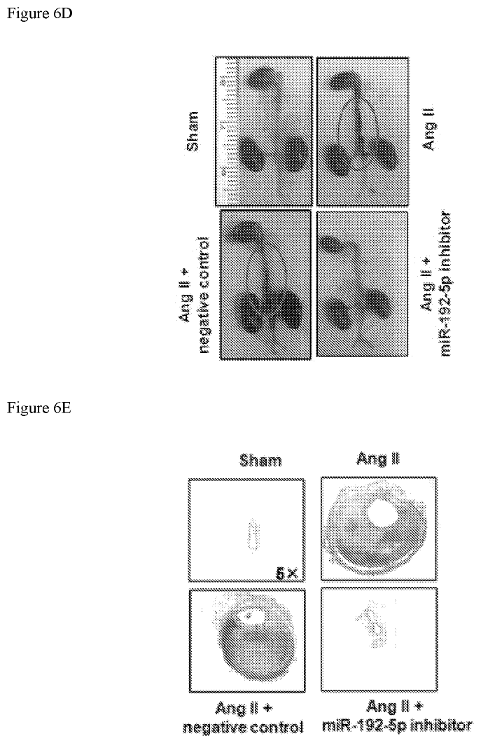

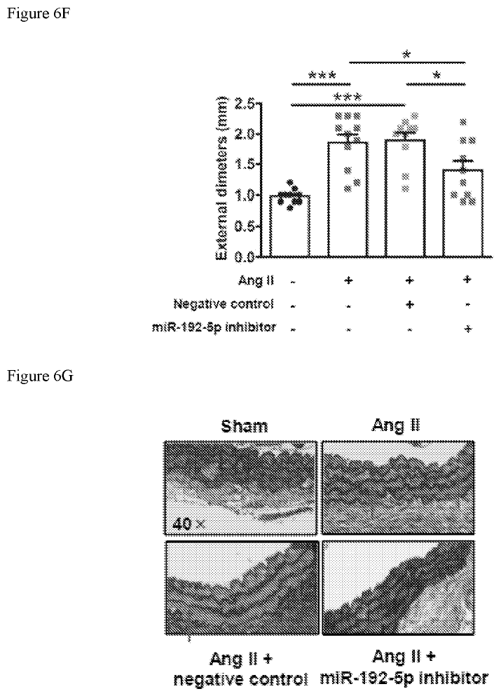

Since miR-192-5p expression was increased in Ang II infused hph-1 mice, the study explored a potential intermediate role of miR-192-5p in the development of AAA via downregulation of DHFR. The incidence of AAA was greatly reduced from 80.0% in negative control treated hph-1 animals to 25.0% in miR-192-5p specific inhibitors treated hph-1 animals after Ang II infusion (FIG. 6A and Table 5). At days 0, 7, and 14, abdominal ultrasound was performed to assess abdominal aorta (AA) dimensions. The sizes of AA measured by echocardiography were significantly smaller in hph-1 mice treated with mmu-miR-192-5p inhibitors, compared to that of control group (FIGS. 6B and 6C). Post-mortem inspection indicated that AAA formation was prevented in Ang II infused hph-1 mice with in vivo treatment of mmu-miR-192-5p inhibitors (FIG. 6D). Representative images of H&E staining were shown in FIG. 6E, which confirmed that mmu-miR-192-5p inhibitors attenuated AAA formation in Ang II infused hph-1 mice. Besides, the external diameters of the AAA were increased in negative control treated hph-1 animals, which were attenuated in miR-192-5p specific inhibitors treated hph-1 mice after Ang II infusion (FIG. 6F). Of note, Ang II infusion induced marked adventitial hypertrophy as well as intra-wall thrombosis, which were markedly attenuated by mmu-miR-192-5p inhibitors. VVG staining indicated significant degradation and flattening of elastic fibers in Ang II infused hph-1 mice, which was substantially abrogated by in vivo treatment with miR-192-5p inhibitors (FIG. 6G). Notably, the sequences of miR-192-5p are the same between human and mouse. Therefore, these results indicate that miR-192-5p inhibitors maybe be readily used as potential therapeutics for human AAA.

Table 5 shows mmu-miR-192-5p specific inhibitors attenuate AAA development in Ang II infused hph-1 mice. Mmu-miR-192-5p-specific inhibitors and negative controls were injected into Ang II infused hph-1 mice, prior to phenotyping of the mice for AAA formation. The actual numbers of animals in each experimental groups with and without AAA. The incidence of AAA was greatly reduced from 80.00% in hph-1 animals treated with negative controls, to 25.00% in hph-1 mice treated with mmu-miR-192-5p specific inhibitors.

TABLE 5

| | AAA | No AAA | Incidence |
|---|---|---|---|
| | (n) | (n) | (%) |
| Sham | 0 | 10 | 0.0 |
| Ang II | 8 | 3 | 72.7 |
| Ang II + negative | 16 | 4 | 80.0 |
| control | | | |
| Ang II + miR-192-5p | 5 | 15 | 25.0 |
| inhibitor | | | |

### DISCUSSION

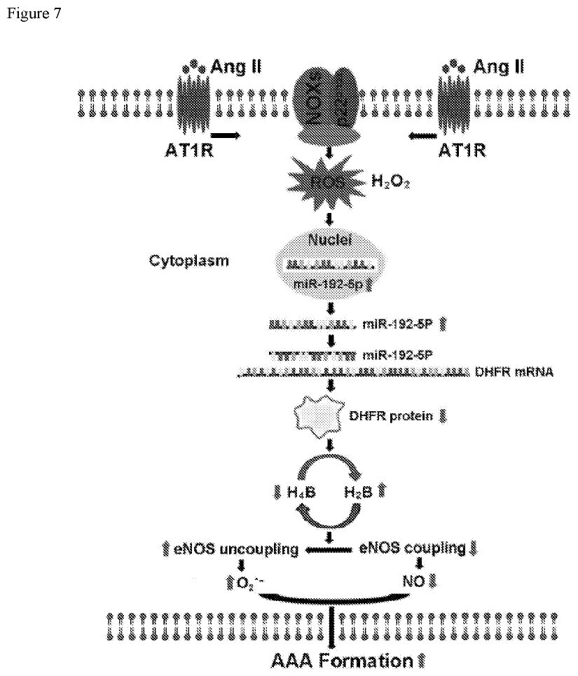

The most significant findings in the present study are the first demonstration that miR-192-5p plays a critical role in mediating NOX-dependent DHFR deficiency in AAA formation, and that in vivo silencing of miR-192-5p expression with specific inhibitors is markedly effective in preventing AAA formation via preservation of endothelial DHFR expression, coupling activity of eNOS and NO bioavailability in ECs. The data indicate that H₂O₂ generated from NOXs activates miR-192-5p expression to reduce DHFR protein abundance, resulting in eNOS uncoupling-dependent AAA formation. Inhibition of miR-192-5p in vivo with specific inhibitors restored endothelial DHFR expression and coupling activity of eNOS to result in reduced oxidative stress, restored NO bioavailability and prevention of matrix degradation and adventitial hypertrophy, hallmarks of AAA formation (FIG. 7). Therefore, miR-192-5p may serve as a novel target for the treatment of AAA.

AAA is a progressive vascular disease, and several miRNAs have been implicated in the pathogenesis of AAA. miR-33a-5p expression in central zone of human AAA is higher than marginal zone, and that miR-33 deletion attenuated AAA formation in mice via downregulation of MMP9 in macrophages and monocyte chemotactic protein-1 in VSMCs. miR-155 expression was found significantly increased in AAA biopsies, while circulating miR-155 levels were also elevated in AAA patients compared with controls, with a 2.67-fold up-regulation at borderline significance. Two immunologically important miR-155 target genes, CTLA4 (cytotoxic T-lymphocyte-associated protein) and SMAD2 (homologies to the Caenorhabditis elegans SMA and MAD family of genes in Drosophila) were found significantly down-regulated within AAA bodies compared with AAA necks, which play an important role in promoting chronic inflammation by enhancing T-cell development and decreasing expression of TGF-β-dependent genes in the nucleus. However, the detailed molecular mechanisms of miRNAs in human AAA need to be explored further, not to mention that the specific roles and regulations of endothelial miRNAs. Here, the data for the first time demonstrated that endothelial miR-192-5p was upregulated in aortic aneurysmal tissues of human AAA, and it played a critical role in mediating AAA formation in Ang II infused hph-1 mice via downregulation of DHFR and consequent uncoupling of eNOS. Inhibition of miR-192-5p in vitro and in vivo restored eNOS coupling activity to result in abrogation of AAA formation.

DHFR deficiency uncouples eNOS to induce hypertension and AAA formation. Relatively modest DHFR deficiency results in a two-fold increase in eNOS uncoupling activity and development of hypertension in Ang II infused WT mice, while more severe DHFR deficiency in Ang II infused hph-1 mice induces a three-fold eNOS uncoupling activity to lead to AAA formation. It was previously established that augmentation of endothelial DHFR expression and activity is robustly effective in protecting against development of AAA. Here, it has been shown for the first time that miR-192-5p was able to induce DHFR deficiency in human endothelial cells in vitro and Ang II infused hph-1 mice in vivo. Besides, miR-192-5p inhibitors restored DHFR mRNA and protein expression in H₂O₂ stimulated endothelial cells and in Ang II-infused hph-1 mice to attenuate eNOS uncoupling activity. Intriguingly, miR-192-5p inhibitors reduced the incidence rate of AAA from 80% to 25%, and substantially attenuated AAA formation at molecular and histological levels in Ang II infused hph-1 mice. The miR-192-5p inhibitors abrogated vascular remodeling including medial elastin degradation and flattening, as well as adventitial hypertrophy, features shown by our previous studies to characterize AAA formation in Ang II infused hph-1 mice. Whether regulation of miR-192-5p in endothelial cells plays a role in adventitial hypertrophy in other vascular diseases remain to be further investigated. Overall, the data establish an important role of miR-192-5p in the formation of AAA in the robust model of Ang II infused hph-1 mice. Notably, the sequences of miR-192-5p are the same between human and mouse. Therefore, miR-192-5p inhibitors maybe are readily used as potential powerful therapeutics for human AAA.

NOX isoform 1, 2 or 4 lies upstream of DHFR deficiency to induce AAA formation. Activation of NOX by Ang II produces ROS to contribute to cardiovascular pathogenesis. NOX produces ROS in response to Ang II in endothelial cells and vascular smooth muscle cells (VSMCs). Endothelial NOX-derived H₂O₂ down-regulates DHFR expression in response to Ang II. Double mutant mice of hph-1-NOX1, hph-1-NOX2, hph-1-p47phox, and hph-1-NOX4 had preserved DHFR expression and activity in endothelial cells in response to Ang II infusion. Of note, miR-192-5p expression was significantly increased in Ang II infused hph-1 mice, which was significantly abrogated in Ang II infused hph-1-NOX1, hph-1-NOX2, hph-1-p47phox and hph-1-NOX4 mice, indicating downstream role of miR-192-5p in mediating NOX-dependent DHFR deficiency. Mmu-miR-192-5p specific inhibitors restored DHFR mRNA and protein expression in Ang II-infused hph-1 mice. Of note, animal and preliminary human data have shown that miRNA mimics and inhibitors have the great potential to develop into a whole new class of therapeutics for the treatment of cardiovascular diseases. miRNAs are small RNA molecules with known sequence that is often remarkably conserved between species, such as miR-192-5p in this research. These characteristics make miRNAs excellent drug targets that can be manipulated with mostly on-target effects, which have promoted miRNA-modulating compounds to enter preclinical efficacy and safety studies as well as in clinical trials. anti-miRs can be generated based on antisense technologies, and can effectively bind to their cognate miRNA targets with excellent affinity and specificity; the mmu-miR-192-5p inhibitors used in this study have indeed proved to be highly efficient and effective in vivo in attenuating AAA formation.

In conclusion, the work represents the first evidence that miR-192-5p, downstream of activation of NOX isoforms in response to Ang II, mediates H₂O₂ induced endothelial DIFR deficiency, eNOS uncoupling and consequent AAA formation. Whereas, specific inhibition of miR-192-5p in Ang II infused hph-1 mice is robustly effective in attenuating AAA formation via preservation of endothelial DHFR expression and eNOS coupling activity, and abrogation of sustained oxidative stress, matrix degradation and vascular remodeling. Since the human and the mouse miR-192-5p sequences are identical, these data indicate that miR-192-5p inhibitors may be readily used as novel therapeutic options for aneurysm (abdominal aortic aneurysm (AAA), thoracic aortic aneurysm (TAA) or cerebral aneurysm), hypertension, ARDS, or any other diseases associated with endothelial dysfunction that can be caused by DHFR deficiency consequent to activation of miR-192-5p.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method of preventing, inhibiting, treating, or reducing aneurysms in a subject, comprising administering to the subject a pharmaceutical composition comprising an miRNA inhibitor comprising a nucleic acid sequence that binds to at least a portion of a miR-192-5p sequence.

2. The method of claim 1, wherein the pharmaceutical composition comprises a vector encoding the miRNA inhibitor.

3. The method of claim 1 or 2, wherein miRNA inhibitor suppresses the function of the mature miR-192-5p.

4. A method of preventing, inhibiting, treating, or reducing aneurysms in a subject, comprising administering to the subject a pharmaceutical composition comprising an miRNA inhibitor comprising a nucleic acid sequence set forth in Tables 1-4.

5. The method of claim 4, wherein the pharmaceutical composition is administered subcutaneously.

6. The method of claim 4, wherein the pharmaceutical composition is administered parenterally.

7. The method of any one of claims 4-6, wherein the aneurysm is abdominal aortic aneurysm, cerebral aneurysm, or thoracic aortic aneurysm.

8. The method of any one of claims 4-7, further comprising conjointly administering to the subject an additional therapeutic agent.

9. The method of claim 8, wherein the additional therapeutic agent is a folate compound, a calcium channel blocker, and/or a reactive oxygen species (ROS) scavenger.

10. A method of reversing vascular remodeling, comprising administering to the subject a pharmaceutical composition comprising an miRNA inhibitor comprising a nucleic acid sequence that binds to at least a portion of a miR-192-5p sequence, wherein vascular remodeling is characterized by inflammation, matrix degradation, adventitial hypertrophy, medial elastin degradation and flattening, and/or formation of intra-lumen thrombi.

11. A method of decreasing reactive oxygen species production, comprising administering to the subject a pharmaceutical composition comprising an miRNA inhibitor comprising a nucleic acid sequence that binds to at least a portion of a miR-192-5p sequence, and wherein the miRNA inhibitor suppresses the function of the mature miR-192-5p.

12. A method of decreasing reactive oxygen species production, comprising administering to the subject a pharmaceutical composition comprising an miRNA inhibitor comprising a nucleic acid sequence set forth in Tables 1-4.

13. A method of restoring endothelial nitric oxide synthase (eNOS) coupling activity, comprising administering to the subject a pharmaceutical composition comprising an miRNA inhibitor comprising a nucleic acid sequence that binds to at least a portion of a miR-192-5p sequence, and wherein the miRNA inhibitor suppresses the function of the mature miR-192-5p.

14. A method of restoring endothelial nitric oxide synthase (eNOS) coupling activity, comprising administering to the subject a pharmaceutical composition comprising a miRNA inhibitor comprising a nucleic acid sequence set forth in Tables 1-4.

15. A method of preserving nitric oxide (NO) bioavailability, comprising administering to the subject a pharmaceutical composition comprising an miRNA inhibitor comprising a nucleic acid sequence that binds to at least a portion of a miR-192-5p sequence, and wherein the miRNA inhibitor suppresses the function of the mature miR-192-5p.

16. A method of preserving nitric oxide (NO) bioavailability, comprising administering to the subject a pharmaceutical composition comprising an miRNA inhibitor comprising a nucleic acid sequence set forth in Tables 1-4.

17. A method of treating or preventing aneurysm (abdominal aortic aneurysm (AAA), thoracic aortic aneurysm (TAA) or cerebral aneurysm), hypertension, acute respiratory distress syndrome (ARDS), or any other diseases associated with endothelial dysfunction in a subject, comprising administering to the subject an miRNA inhibitor comprising a nucleic acid that is at least 50-100% identical to any one of SEQ ID NOs: 1-19.

18. A method of treating or preventing aneurysm (abdominal aortic aneurysm (AAA), thoracic aortic aneurysm (TAA) or cerebral aneurysm), hypertension, acute respiratory distress syndrome (ARDS), or any other diseases associated with endothelial dysfunction in a subject, comprising administering to the subject a miRNA inhibitor comprising a nucleic acid sequence that binds to at least a portion of a miR-192-5p sequence.

19. The method of claim 18, wherein the miRNA inhibitor has at least 50% complementarity to a portion of a miR-192-5p sequence.

20. The method of claim 18, wherein the miRNA inhibitor has at least 95% complementarity to a portion of a miR-192-5p sequence.

21. The method of claim 18, wherein the miRNA inhibitor has at least 99% complementarity to a portion of a miR-192-5p sequence.

22. The method of claim 18, wherein the miRNA inhibitor has 100% complementarity to a portion of a miR-192-5p sequence.

23. The method of any one of claims 18-22, wherein the miRNA inhibitor suppresses the function of the mature miR-192-5p.

24. The method of any one of claims 1-23, wherein the miRNA inhibitor comprises a nucleic acid sequence that is at least 50% identical to any one of SEQ ID NOs: 1-19.

25. The method of any one of claims 1-23, wherein the miRNA inhibitor comprises a nucleic acid sequence that is at least 90% identical to any one of SEQ ID NOs: 1-19.

26. The method of any one of claims 1-23, wherein the miRNA inhibitor comprises a nucleic acid sequence that is at least 95% identical to any one of SEQ ID NOs: 1-19.

27. The method of any one of claims 1-23, wherein the miRNA inhibitor comprises a nucleic acid sequence that is at least 98% identical to any one of SEQ ID NOs: 1-19.

28. The method of any one of claims 1-23, wherein the miRNA inhibitor comprises a nucleic acid sequence that is any one of SEQ ID NOs: 1-19.

29. The method of any one of claims 24-28, wherein the nucleic acid comprises a chemical modification.

30. The method of claim 29, wherein the chemical modification is a 2′-O-methylated nucleoside (2′OMe), a 2′-fluoro oligonucleotide (2′F), a 2′-O-methoxyethyl oligonucleotide (2′MOE), a phosphorodiamidate morpholino oligonucleotide (PMO), a peptide nucleic acid (PNA), a phosphorothioate bond (PS), a locked nucleic acid (LNA), a non-nucleotide N,N-diethyl-4-(4-nitronaphthalen-1-ylazo)-phenylamine (ZEN), a hydrophobic moiety, a naphthyl modifier, or a cholesterol moiety.

31. The method of claim 30, wherein the chemical modification is a 2′-O-methylated nucleoside (2′OMe).

32. The method of claim 30, wherein the chemical modification is a 2′-fluoro oligonucleotide (2′F).

33. The method of claim 30, wherein the chemical modification is a 2′-O-methoxyethyl oligonucleotide (2′MOE).

34. The method of claim 30, wherein the chemical modification is a phosphorodiamidate morpholino oligonucleotide (PMO).

35. The method of claim 30, wherein the chemical modification is a peptide nucleic acid (PNA).

36. The method of claim 30, wherein the chemical modification is a phosphorothioate bond (PS).

37. The method of claim 30, wherein the chemical modification is a locked nucleic acid (LNA).

38. The method of claim 30, wherein the chemical modification is a hydrophobic moiety.

39. The method of claim 30, wherein the chemical modification is a naphthyl modifier.

40. The method of claim 30, wherein the chemical modification is a cholesterol moiety.

41. The method of claim 30, wherein the chemical modification is a non-nucleotide N,N-diethyl-4-(4-nitronaphthalen-1-ylazo)-phenylamine (ZEN).

42. The method of any one of claims 24-41, wherein the nucleic acid is complementary to any one of SEQ ID NOs: 20-38.

43. The method of any one of claims 24-42, wherein the miRNA inhibitor binds to a miRNA comprising a nucleic acid that is at least 50% identical to any one SEQ ID NOs: 20-38.

44. The method of any one of claims 24-42, wherein the miRNA inhibitor binds to a miRNA comprising a nucleic acid that is at least 90% identical to any one SEQ ID NOs: 20-38.

45. The method of any one of claims 24-42, wherein the miRNA inhibitor binds to a miRNA comprising a nucleic acid that is at least 95% identical to any one SEQ ID NOs: 20-38.

46. The method of any one of claims 24-42, wherein the miRNA inhibitor binds to a miRNA comprising a nucleic acid that is at least 98% identical to any one SEQ ID NOs: 20-38.

47. The method of any one of claims 24-42, wherein the miRNA inhibitor binds to a miRNA comprising a nucleic acid that is any one SEQ ID NOs: 20-38.

48. The method of any one of claims 24-47, wherein the miRNA inhibitor is at least 5 nucleotides in length.

49. The method of any one of claims 24-47, wherein the miRNA inhibitor is at least 18 nucleotides in length.

50. The method of any one of claims 24-49, wherein the miRNA inhibitor is no more than 35 nucleotides in length.

51. The method of any one of claims 24-49, wherein the miRNA inhibitor is no more than 22 nucleotides in length.
